(19) United States
(12) Patent Application Publication
(10) Pub. No.: US 2014/0065649 A1
(43) Pub. Date: Mar. 6, 2014

(54) TEST SYSTEMS AND METHODS FOR IDENTIFYING AND CHARACTERISING LIPID LOWERING DRUGS

(71) Applicants: Hans-Ludwig Schaefer, Franfurt (DE); Eugen Falk, Frankfurt (DE); Uwe Schwahn, Frankfurt (DE)

(72) Inventors: Hans-Ludwig Schaefer, Franfurt (DE); Eugen Falk, Frankfurt (DE); Uwe Schwahn, Frankfurt (DE)

(73) Assignee: Sanofi, Paris (FR)

(21) Appl. No.: 14/114,523

(22) PCT Filed: Apr. 30, 2012
(86) PCT No.: PCT/EP12/57890

§ 371 Date: Oct. 28, 2013

(30) Foreign Application Priority Data
Apr. 29, 2011 (EP) 11305513.1
Apr. 29, 2011 (EP) 11305514.9
Jan. 27, 2012 (EP) 1215286.24

(51) Int. Cl. A61K 49/00

(52) U.S. Cl. CPC A61K 49/0008
USPC 435/11

(57) ABSTRACT

The present invention relates to methods for the identification and characterization of therapeutic candidates for use in the treatment of a disease or condition associated with elevated LDL-C levels involving a rodent, methods for the testing of the efficacy of an antibody specifically binding to proprotein convertase subtilisin/kexin type 9 (PCSK9) involving a rodent, as well as a rodent and its use in the identification or profiling of compounds for modulation of a disease or condition associated with elevated LDL-C levels.

The present invention relates to methods for the identification and characterization of therapeutic candidates for use in the treatment of a disease or condition associated with elevated LDL-C levels involving a rodent, methods for the testing of the efficacy of an antibody specifically binding to proprotein convertase subtilisin/kexin type 9 (PCSK9) involving a rodent, as well as a rodent and its use in the identification or profiling of compounds for modulation of a disease or condition associated with elevated LDL-C levels.

The present invention also relates to kits and articles of manufacture comprising packaging material and a rodent and optionally PCSK9-specific antibodies or antigen-binding fragments thereof, and a label or packaging insert, for conducting the above methods.

### BACKGROUND OF THE INVENTION

Proprotein convertase subtilisin/kexin type 9 (PCSK9) is a proprotein convertase belonging to the proteinase K subfamily of the secretory subtilase family. The encoded protein is synthesized as a soluble zymogen that undergoes autocatalytic intramolecular processing in the endoplasmic reticulum. Evidence suggest that PCSK9 increases plasma LDL cholesterol by promoting degradation of the LDL receptor, which mediates LDL endocytosis in the liver, the major route of LDL clearance from circulation. The structure of PCSK9 protein shows that it has a signal sequence, followed by a prodomain, a catalytic domain that contains a conserved triad of residues (D186, H226 and S386), and a C-terminal domain. It is synthesized as a soluble 74-kDa precursor that undergoes autocatalytic cleavage in the ER, generating a 14-kDa prodomain and 60-kDa catalytic fragment. The autocatalytic activity has been shown to be required for secretion. After cleavage the prodomain remains tightly associated with the catalytic domain.

Antibodies to PCSK9 are described in, for example, WO 2008/057457, WO 2008/057458, WO 2008/057459, WO 2008/063382, WO 2008/125623, and US 2008/0008697. Anti-PCSK9 antibodies that are particularly well-suited for practicing the present invention are disclosed in US 2010/0166768 A1, the content of which is hereby incorporated by reference in its entirety.

### TECHNICAL PROBLEMS UNDERLYING THE PRESENT INVENTION

Cardiovascular diseases (CVDs) are a leading cause of morbidity and mortality, worldwide, accounting for approximately 30% of all deaths in the USA and almost 50% of all deaths in Europe. Numerous studies have demonstrated a direct correlation between the level of low-density lipoprotein cholesterol (LDL-C) and the rate of CV events. A meta-analysis of 20 randomized clinical trials, for example, showed that each 1 mmol/L (40 mg/dL) reduction in LDL-C is associated with a significant 22% reduction in CVD morbidity and mortality. Based on the results from this and similar studies, international treatment guidelines recommend lowering LDL-C to <2.0-2.6 mmol/L (<77-100 mg/dL) in patients with established CVDs and to <1.8-2.0 mmol/L (<70-77 mg/dL) in high-risk groups such as those with CVDs plus diabetes, smoking, poorly controlled hypertension, metabolic syndrome, or previous myocardial infarction. However, despite the widespread availability of lipid-lowering agents, approximately 30% of all adult patients treated for hypercholesterolemia in the United States between 1999 and 2006 failed to achieve their recommended LDL-C targets. Reasons for this include poor adherence to therapy, drug-resistance/intolerance and the positive relationship between adverse event rates and increasing dosage. Moreover, since the most effective lipid-lowering agents can only reduce LDL-C levels by up to 55%, target attainment rates in patients that require substantial reductions in LDL-C, such as those with familial hypercholesterolemia, are often significantly lower than might be expected. More effective lipid-lowering agents are therefore required to improve target attainment rates in these patients.

Cholesterol homeostasis involves 3 major rate-determining steps: cholesterol synthesis, secretion and removal from the circulation. The majority of lipid-lowering agents primarily target cholesterol synthesis and/or secretion. However, a new class of lipid-lowering drug—the proprotein convertase subtilisin/kexin type 9 (PCSK9) inhibitors—is being developed that reduces LDL-C levels by increasing the rate of cholesterol uptake from the circulation via the LDL receptor (LDLR).

PCSK9 is the ninth member of the subtilisin family of serine proteases. It is mainly expressed in the liver and intestine and is modulated by a series of transcription factors, the most important of which is SREBP-2. Under normal conditions, PCSK9 binds to the extracellular epidermal growth factor-A domain of the LDLR and is internalized by endocytosis. A decrease in endosomal pH increases PCSK9's affinity for the LDLR and the entire complex is targeted to the lysosome for degradation. The increased rate of LDLR degradation reduces the rate of LDL-C removal from the circulation, thereby increasing the serum level of LDL-C. It therefore follows that reducing the ratio between PCSK9 to LDLR and/or disrupting the interaction between the 2 proteins can potentially reduce LDL-C levels in patients with hypercholesterolemia. Moreover, PSCK9 inhibitors have the potential to increase the efficacy of statins. Cholesterol depletion activates SREBP-2 translocation, causing it to bind to the SRE-1 element of the PCSK9 promoter. Because an SRE-1 motif is present in both the LDLR and the PCSK9 promoters, lipid-lowering using statins increases transcription of both LDLR and its natural inhibitor, PCSK9. This feedback loop is thought to limit the LDL-C-lowering efficacy of statins at higher doses. An effective PCSK9 inhibitor that provides additional LDL-C reductions, either alone or in combination with a statin, is therefore likely to have a profound impact on the future management of CVD.

Statins are among the most widely used drugs in the world. Although statins generally exhibit an excellent safety profile, it is desirable to further optimize the safety profile by reducing the already low rate of unwanted side-effects (such as myopathies). In order to find alternative treatment regimes, biological test systems are necessary in order to screen potential drug candidates for their activity and potential adverse effects in advance to their use in humans.

Test systems based on rodents and rodent cells are widely used for drug screening of small molecule and biological molecule drug candidates. As concerns HMG-CoA reductase inhibitors such as statins, however, it has previously been found that these are not effective in rodents such as hamster.

Quite surprisingly, the inventors of the present invention found that the administration of anti-PCSK9 antibodies or fragments thereof leads to a lowering of the LDL-cholesterol levels in syrian hamsters. Moreover, there was a synergistic effect of HMG-CoA reductase inhibitor treatment when applied together with the antibody treatment although HMG-CoA reductase inhibitor treatment alone had no effect on LDL-C levels.

This surprising finding allows for the use of said mammals for the development of biological test systems for testing of potentially new drugs effective for the treatment and/or prevention of cardiovascular diseases.

The above overview does not necessarily describe all problems solved by the present invention.

### SUMMARY OF THE INVENTION

This summary of the invention does not necessarily describe all features of the present invention. Other embodiments will become apparent from a review of the ensuing detailed description.

In a first aspect, present invention is directed to method for screening compounds to identify therapeutic candidates for the modulation of a disease or condition associated with elevated LDL-C levels, said method comprising:
(a) providing a rodent
(b) administering a test compound to the rodent and
(c) detecting whether said compound increases or decreases one or more parameters selected from the group consisting of: total cholesterol (TC), low-density cholesterol (LDL-C) and high-density cholesterol (HDL-C) in said rodent in comparison to a control rodent;
   wherein a modulation of one or more of said parameters indicates that said compound is a candidate for modulating said disease or condition in vivo.

In a second aspect, present invention is directed to a method for screening compounds to identify therapeutic candidates for the modulation of a disease or condition associated with elevated LDL-C level, said method comprising:
(a) providing a rodent
(b) administering a test compound to the rodent
(c) determining one or more parameters of the rodent selected from the group consisting of: total cholesterol (TC), low-density cholesterol (LDL-C) and high-density cholesterol (HDL-C) before treatment of the rodent with the compound
(d) determining the one or more parameters after treatment of the rodent with the compound, and
(e) comparing the results obtained in (a) with those obtained in (b),
   wherein a difference of the parameters of (a) in comparison with those of (b) indicates that the compound is a candidate for modulating said disease or condition in vivo.

In a third aspect present invention concerns a method of testing the efficacy of an antibody or an antigen-binding fragment thereof which specifically binds to hPCSK9 for the treatment of a disease or condition associated with elevated LDL-C levels, said method comprising:
(a) administering said antibody to a rodent; and
(b) determining the total cholesterol, LDL-C or HDL-C level of the rodent before and after administration of said antibody or antigen-binding fragment thereof to the rodent,
   wherein a reduction of the total cholesterol and/or LDL-C level and/or a increase of the HDL-C level determined after administration of the antibody relative to the predose level determined before administration of the antibody is indicative that the antibody or antigen-binding fragment thereof is efficacious for the treatment of said disease or condition, and
   wherein the increase of the total cholesterol level and/or the LDL-C level determined after administration of the antibody relative to the predose level determined before administration of the antibody is indicative that the antibody exhibits adverse effects in promoting, contributing to or triggering said disease or condition in vivo.

In a fourth aspect, present invention concerns a method of testing the efficacy of an antibody or an antigen-binding fragment thereof which specifically binds hPCSK9 for the modulation of a disease or condition associated with elevated LDL-C levels, said method comprising:
(a) determining the total cholesterol level, the LDL-C level and/or the HDL-level in an in vitro sample obtained from a rodent before treatment of the rodent with the antibody,
(b) determining the total cholesterol level, the LDL-C level and/or the HDL-level in an in vitro sample obtained from the rodent after treatment of the rodent with the antibody, and
(c) comparing the results obtained in (a) with those obtained in (b),
   wherein a reduction of the total cholesterol and/or LDL-C level and/or a increase of the HDL-C level determined in (b) relative to the predose level determined in (a) before administration of the antibody is indicative that the antibody or antigen-binding fragment thereof is efficacious for the treatment and/or prevention of said disease or condition, and
   wherein the increase of the total cholesterol level and/or the LDL-C level in (b) in comparison the predose level detected in (a) is indicative that the antibody exhibits adverse effects in promoting, contributing to or triggering said disease or condition in vivo.

In a fifth aspect, present invention concerns a method of testing the efficacy of an antibody or an antigen-binding fragment thereof which specifically binds hPCSK9 for the treatment of a disease or condition associated with elevated LDL-C levels, said method comprising:
(a) administering said antibody to a rodent; and
(b) determining the efficacy of said antibody or antigen-binding fragment thereof by determining the total cholesterol level and/or LDL-C level and/or HDL-C level of the rodent after administration of said antibody or antigen-binding fragment thereof,
(c) determining the total cholesterol level and/or LDL-C level and/or HDL-C level of a control rodent that has not been treated with the antibody and has preferably obtained a placebo,
   wherein the antibody is considered efficacious for the treatment of the disease or condition if the total cholesterol level and/or LDL-C level determined in (c) is lower and/or the HDL-C level determined in (c) is higher than that determined in (b) and
   wherein the antibody is considered to exhibit adverse effects if the total cholesterol level and/or LDL-C level determined in (c) is higher than that determined in (b).

In a sixth aspect, present invention concerns an in vitro method of testing the efficacy of an antibody or an antigen-binding fragment thereof which specifically binds hPCSK9 for the treatment of a disease or condition associated with elevated LDL-C levels, said method comprising:
(a) determining the total cholesterol level and/or LDL-C level and/or HDL-C level in a sample of a rodent obtained after administration of said antibody or antigen-binding fragment thereof to the rodent,
(b) determining the total cholesterol level and/or LDL-C level and/or HDL-C level in a control sample obtained from a rodent that has not been treated with said antibody or antigen-binding fragment thereof,
   wherein the antibody is considered efficacious for the treatment of the disease or condition if the total cholesterol level and/or LDL-C level determined in (b) is lower and/or the HDL-C level determined in (b) is higher than that determined in (a) and
   wherein the antibody is considered to exhibit adverse effects if the total cholesterol level and/or LDL-C level determined in (b) is higher than that determined in (a).

In a seventh aspect, present invention concerns a method for testing the efficacy of a compound in modulating cholesterol levels in a subject, comprising the steps:
(a) providing a rodent;
(b) administering an antibody or an antigen-binding fragment thereof which specifically binds PCSK9 to the rodent;
(c) administering a test compound to said rodent;
(d) determining one or more parameters of the rodent selected from the group consisting of: the total cholesterol level, LDL-C level or HDL-C level, after administration of the test compound,
(e) determining the same one or more parameter(s) of a control rodent that has not been challenged with the test compound
   wherein a difference in the cholesterol (total or LDL-C or HDL-C) determined in (a) and determined in (b) indicates that the test compound is efficacious in modulating cholesterol levels in a subject.

In an eighth aspect, present invention concerns an in vitro method for testing the efficacy of a compound in modulating cholesterol levels in a subject, comprising the steps:
(a) determining in a sample of a rodent taken after the rodent has been applied a test compound one or more of the parameters selected from the group consisting of: the total cholesterol level, LDL-C level or HDL-C level,
(b) determining the same one or more parameter(s) in a sample of a control rodent that has not been challenged with the test compound
   wherein both animals have been administered an antibody or an antigen-binding fragment thereof which specifically binds PCSK9 in addition to the test compound, and
   wherein a difference in the cholesterol (total C and/or LDL-C and/or HDL-C) determined in (a) and determined in (b) indicates that the test compound is efficacious in modulating cholesterol levels in a subject.

In a ninth aspect, present invention concerns a method for testing the efficacy of a compound in modulating cholesterol levels in a subject, comprising the steps:
(a) providing a rodent;
(b) administering an antibody or an antigen-binding fragment thereof which specifically binds PCSK9 to the rodent;
(c) administering a test compound to said rodent;
(d) determining in the rodent one or more of the parameters selected from the group consisting of: total cholesterol levels, LDL-C levels or HDL-C levels
   (i) before administration of the test compound to the rodent and
   (ii) after administration of the test compound to the rodent:,
(e) comparing the parameters obtained in (d)(ii) and (d)(ii)
   wherein a difference in the parameters obtained in (d) (ii) with the parameter obtained in (d) (ii) indicates that the test compound compound is efficacious in modulating cholesterol levels in a subject.

In a tenth aspect, present invention concerns an in vitro method for testing the efficacy of a compound in modulating cholesterol levels in a subject, comprising the steps:
(a) determining one or more of the parameters selected from the group consisting of: total cholesterol levels, LDL-C levels or HDL-C levels,
   (i) in a sample of a rodent obtained before administration of the test compound to the rodent, and
   (ii) in a sample of the same rodent obtained administration of the test compound, and
(b) comparing the parameters determined in (d)(ii) and (d)(ii)
   wherein the rodent has been administered an antibody or an antigen-binding fragment thereof which specifically binds PCSK9 in conjunction with the test administration compound and
   wherein a difference in the parameters obtained in (d) (ii) with the parameter obtained in (d) (ii) indicates that the test compound compound is efficacious in modulating cholesterol levels in a subject.

In an eleventh aspect, present invention is directed to a rodent for use in identifying a drug for the treatment of a disease associated with elevated cholesterol levels and preferably associated with elevated LDL-C levels, wherein the rodent has decreased PCSK9 levels in comparison to a control rodent.

In a twelfth aspect, present invention is directed to the use of a rodent with decreased PCSK9 levels in comparison to a control rodent as a model system for determining the cholesterol-modulating effect and preferably of the cholesterol-lowering effect of a drug. In a preferred embodiment of the eleventh or twelfth aspect, the drug is an HMG-CoA reductase inhibitor such as a statin.

In a thirteenth aspect, present invention concerns a method for the preparation of a rodent suitable for use as model system for determining the cholesterol-modulating effect and preferably of the cholesterol-lowering effect of a drug, the method comprising providing a rodent or a blastocyst of a rodent and lowering its PCSK9 level by means of a genomic knock-out of PCSK9, a stable or transient knock-down of PCSK9 or administration of a PCSK-9 antagonist.

In a fourteenth aspect, present invention concerns a rodent, preferably a hamster, obtained by a method according to the thirteenth aspect, and preferably obtained by administration of a PCSK-9 specific antibody to the rodent.

In a fifteenth aspect, present invention concerns a kit for conducting a method according to one of the aspects 1 to 10 comprising a rodent, preferably a hamster and a PCSK 9-specific antagonist, such as a PCSK9-specific antibody and optionally comprising one or more of the further components according to one of the aspects sixteen and seventeen.

In a sixteenth aspect, present invention concerns an article of manufacture comprising
(a) a packaging material or container;
(b) an antibody or an antigen-binding fragment thereof which specifically binds hPCSK9; and
(c) a data carrier such as a label or packaging insert contained within the packaging material containing instructions for carrying out a method according to one of aspects 1 to 10 for profiling or identifying compounds for use in the treatment or prevention of hypercholesterolemia, hyperlipidemia, dyslipidemia, atherosclerosis and cardiovascular diseases and optionally
(d) one or more buffers and/or reagents for determining total cholesterol levels, LDL-C levels or HDL-C levels in a sample.

In a seventeenth aspect, present invention concerns article of manufacture comprising
(a) a packaging material or container;
(b) reagents and buffers for determining total cholesterol levels, LDL-C levels or HDL-C levels in a sample; and
(c) a data carrier such as a label containing instructions for carrying out a method according to one of the aspects 1 to 10 and optionally
(d) an antibody or an antigen-binding fragment thereof which specifically binds hPCSK9.

### BRIEF DESCRIPTION OF THE DRAWINGS

FIG. 1. Changes in mean serum A: Low-density lipoprotein-cholesterol; B: Total cholesterol and C: Triglyceride in male Syrian hamsters on a normal chow diet after a single s.c. injection of 316P 1, 3, or 10 mg/kg or PBS (control) in Study 1. Each data point represents the mean±SEM (n=6) for each time point.

^{#}P≦0.05 2-way ANOVA followed by the Dunnett test for naive control vs PBS control.

*P≦0.05 2-way ANOVA followed by the Dunnett test for treated groups vs PBS control.

FIG. 2. Changes in mean serum A. Low-density lipoprotein-cholesterol; B. Total cholesterol and C. Triglyceride in male Syrian hamsters on a normal chow diet receiving a twice daily p.o. dose of atorvastatin 10 mg/kg or 20 mg/kg for 7 days followed by a single s.c. injection of 316P 10 mg/kg or PBS (control) in Study 2. Each data point represents the mean±SEM (n=10) for each time point.

FIG. 3. Mean 316P (hFc) levels in male Syrian hamsters on a normal chow diet after a single s.c. injection of 316P 1, 3, or 10 mg/kg or PBS (control) in Study 1. Each data point represents the mean±SEM (n=6) for each time point.

FIG. 4. Western blot analysis showing relative levels of hepatic low density lipoprotein receptor (LDLR) protein in normolipidemic hamsters treated with PBS s.c. (control), 316P 10 mg/kg, atorvastatin 2×20 mg/kg, 316P+atorvastatin or PBS p.o. control.

FIG. 5: Sequences related to PCSK9, wherein FIG. 5a) shows the amino acid sequence of PCSK9 of chinese hamster according to GenBank accession number XP_{—}00349578.1 (SEQ ID NO:1), FIG. 5b) shows the amino acid sequence of human PCSK9 (SEQ ID NO:2). The protein sequence of human PCSK9 can further be retrieved from the NCBI database under the reference number NP_{—}777596 (e.g. NP_{—}777596.2). FIG. 5c) shows the nucleic acid sequence of PCSK9 mRNA of chinese hamster (SEQ ID NO:11) according to GenBank accession number XM_{—}003495737. Sequences can be retrieved from the NCBI Database under the link http://www.ncbi.nlm.nih.gov/ by means of the accession number.

FIG. 6: Sequences related to antibody 316P used in present invention showing SEQ ID Nos 3, 4, 5, 6, 7, 8, 9 and 10. Further details relating to these sequences are described in the section “Preferred antibodies for practicing present invention”.

FIG. 7: Sequences related to antibody 300N, a further preferred antibody for use in present invention, showing SEQ ID Nos 12, 13, 14, 15, 16, 17, 18 and 19. Further details relating to these sequences are described in the section “Preferred antibodies for practicing present invention”.

### DETAILED DESCRIPTION OF THE INVENTION

### Definitions

Before the present invention is described in detail below, it is to be understood that this invention is not limited to the particular methodology, protocols and reagents described herein as these may vary. It is also to be understood that the terminology used herein is for the purpose of describing particular embodiments only, and is not intended to limit the scope of the present invention which will be limited only by the appended claims. Unless defined otherwise, all technical and scientific terms used herein have the same meanings as commonly understood by one of ordinary skill in the art to which this invention belongs.

Preferably, the terms used herein are defined as described in “A multilingual glossary of biotechnological terms: (IUPAC Recommendations)”, Leuenberger, H. G. W, Nagel, B. and Kölbl, H. eds. (1995), Helvetica Chimica Acta, CH-4010 Basel, Switzerland).

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integer or step.

Several documents (for example: patents, patent applications, scientific publications, manufacturer's specifications, instructions, GenBank Accession Number sequence submissions etc.) are cited throughout the text of this specification. Nothing herein is to be construed as an admission that the invention is not entitled to antedate such disclosure by virtue of prior invention. Some of the documents cited herein are characterized as being “incorporated by reference”. In the event of a conflict between the definitions or teachings of such incorporated references and definitions or teachings recited in the present specification, the text of the present specification takes precedence.

Sequences: All sequences referred to herein are disclosed in the attached sequence listing that, with its whole content and disclosure, is a part of this specification.

The term “about” when used in connection with a numerical value is meant to encompass numerical values within a range having a lower limit that is 5% smaller than the indicated numerical value and having an upper limit that is 5% larger than the indicated numerical value.

The term “human proprotein convertase subtilisin/kexin type 9” or “hPCSK9”, as used herein, refers to hPCSK9 having the nucleic acid sequence shown in SEQ ID NO: 754 of patent application US 2010/0166768 A1 that is incorporated herein by reference in its entirety (see above) and the amino acid sequence of SEQ ID NO: 2, or a biologically active fragment thereof.

The terms “specifically binds”, “specific binding” or the like, mean that an antibody or antigen-binding fragment thereof forms a complex with an antigen that is relatively stable under physiologic conditions. Specific binding can be characterized by an equilibrium dissociation constant of at least about 1×10⁻⁶ M or less (e.g., a smaller K_{D} denotes a tighter binding). Methods for determining whether two molecules specifically bind are well known in the art and include, for example, equilibrium dialysis, surface plasmon resonance, and the like. An isolated antibody that specifically binds hPCSK9 may, however, exhibit cross-reactivity to other antigens such as PCSK9 molecules from other species. Moreover, multi-specific antibodies (e.g., bispecifics) that bind to hPCSK9 and one or more additional antigens are nonetheless considered antibodies that “specifically bind” hPCSK9, as used herein.

The term “K_{D}”, as used herein, is intended to refer to the equilibrium dissociation constant of a particular antibody-antigen interaction. The equilibrium dissociation constant is typically measured in “mol/L” (abbreviated as “M”).

By the term “slow off rate”, “Koff” or “kd” is meant an antibody that dissociates from hPCSK9 with a rate constant of 1×10⁻³ s⁻¹ or less, preferably 1×10⁻⁴ s⁻¹ or less, as determined by surface plasmon resonance, e.g., BIACORE™.

The term “high affinity” antibody refers to those mAbs having a binding affinity to hPCSK9 of at least 10⁻¹⁰ M; preferably 10⁻¹¹ M; even more preferably 10⁻¹² M, as measured by surface plasmon resonance, e.g., BIACORE™ or solution-affinity ELISA.

The term “surface plasmon resonance”, as used herein, refers to an optical phenomenon that allows for the analysis of real-time biospecific interactions by detection of alterations in protein concentrations within a biosensor matrix, for example using the BIACORE™ system (Pharmacia Biosensor AB, Uppsala, Sweden and Piscataway, N.J.).

An “epitope”, also known as antigenic determinant, is the region of an antigen that is recognized by the immune system, specifically by antibodies, B cells, or T cells. As used herein, an “epitope” is the part of an antigen capable of binding to an antibody or antigen-binding fragment thereof as described herein. In this context, the term “binding” preferably relates to a “specific binding”, as defined herein. Epitopes usually consist of chemically active surface groupings of molecules such as amino acids, sugar side chains, phosphoryl groups, or sulfonyl groups and may have specific three-dimensional structural characteristics and/or specific charge characteristics. Conformational and non-conformational epitopes can be distinguished in that the binding to the former but not the latter is lost in the presence of denaturing solvents.

A “paratope” is the part of an antibody that specifically binds to the epitope.

The term “antibody”, as used herein, is intended to refer to immunoglobulin molecules comprised of four polypeptide chains, two heavy (H) chains and two light (L) chains inter-connected by disulfide bonds. The term “antibody” also includes all recombinant forms of antibodies, in particular of the antibodies described herein, e.g. antibodies expressed in prokaryotes, unglycosylated antibodies, and any antigen-binding antibody fragments and derivatives as described below. Each heavy chain is comprised of a heavy chain variable region (“HCVR” or “VH”) and a heavy chain constant region (comprised of domains CH1, CH2 and CH3). Each light chain is comprised of a light chain variable region (“LCVR or “VL”) and a light chain constant region (CL). The VH and VL regions can be further subdivided into regions of hypervariability, termed complementarity determining regions (CDR), interspersed with regions that are more conserved, termed framework regions (FR). Each VH and VL is composed of three CDRs and four FRs, arranged from amino-terminus to carboxy-terminus in the following order: FR1, CDR1, FR2, CDR2, FR3, CDR3, FR4. The variable regions of the heavy and light chains contain a binding domain that interacts with an antigen. The constant regions of the antibodies may mediate the binding of the immunoglobulin to host tissues or factors, including various cells of the immune system (e.g., effector cells) and the first component (C1q) of the classical complement system.

Substitution of one or more CDR residues or omission of one or more CDRs is also possible. Antibodies have been described in the scientific literature in which one or two CDRs can be dispensed with for binding. Padlan et al. (1995 FASEB J. 9:133-139) analyzed the contact regions between antibodies and their antigens, based on published crystal structures, and concluded that only about one fifth to one third of CDR residues actually contact the antigen. Padlan also found many antibodies in which one or two CDRs had no amino acids in contact with an antigen (see also, Vajdos et al. 2002 J Mol Biol 320:415-428).

CDR residues not contacting antigen can be identified based on previous studies (for example residues H60-H65 in CDRH2 are often not required), from regions of Kabat CDRs lying outside Chothia CDRs, by molecular modeling and/or empirically. If a CDR or residue(s) thereof is omitted, it is usually substituted with an amino acid occupying the corresponding position in another human antibody sequence or a consensus of such sequences. Positions for substitution within CDRs and amino acids to substitute can also be selected empirically. Empirical substitutions can be conservative or non-conservative substitutions.

The term “antigen-binding fragment” of an antibody (or simply “binding portion”), as used herein, refers to one or more fragments of an antibody that retain the ability to specifically bind to its antigen, such as hPCSK9. It has been shown that the antigen-binding function of an antibody can be performed by fragments of a full-length antibody. Examples of binding fragments encompassed within the term “antigen-binding fragment” of an antibody include (i) Fab fragments, monovalent fragments consisting of the VL, VH, CL and CH domains; (ii) F(ab′)₂ fragments, bivalent fragments comprising two Fab fragments linked by a disulfide bridge at the hinge region; (iii) Fd fragments consisting of the VH and CH domains; (iv) Fv fragments consisting of the VL and VH domains of a single arm of an antibody, (v) dAb fragments (Ward et al., (1989) Nature 341: 544-546), which consist of a VH domain; (vi) isolated complementarity determining regions (CDR), and (vii) combinations of two or more isolated CDRs which may optionally be joined by a synthetic linker. Furthermore, although the two domains of the Fv fragment, VL and VH, are coded for by separate genes, they can be joined, using recombinant methods, by a synthetic linker that enables them to be made as a single protein chain in which the VL and VH regions pair to form monovalent molecules (known as single chain Fv (scFv); see e.g., Bird et al. (1988) Science 242: 423-426; and Huston et al. (1988) Proc. Natl. Acad. Sci. USA 85: 5879-5883). Such single chain antibodies are also intended to be encompassed within the term “antigen-binding fragment” of an antibody. A further example is a binding-domain immunoglobulin fusion protein comprising (i) a binding domain polypeptide that is fused to an immunoglobulin hinge region polypeptide, (ii) an immunoglobulin heavy chain CH2 constant region fused to the hinge region, and (iii) an immunoglobulin heavy chain CH3 constant region fused to the CH2 constant region. The binding domain polypeptide can be a heavy chain variable region or a light chain variable region. The binding-domain immunoglobulin fusion proteins are further disclosed in US 2003/0118592 and US 2003/0133939. These antibody fragments are obtained using conventional techniques known to those with skill in the art, and the fragments are screened for utility in the same manner as are intact antibodies. Further examples of “antigen-binding fragments” are so-called microantibodies, which are derived from single CDRs. For example, Heap et al. describe a 17 amino acid residue microantibody derived from the heavy chain CDR3 of an antibody directed against the gp120 envelope glycoprotein of HIV-1 (Heap C J et al. (2005) J. Gen. Virol. 86:1791-1800). Other examples include small antibody mimetics comprising two or more CDR regions that are fused to each other, preferably by cognate framework regions. Such a small antibody mimetic comprising VH CDR1 and VL CDR3 linked by the cognate VH FR2 has been described by Qiu et al. (Qiu X-Q, et al. (2007) Nature biotechnology 25 (8): 921-929).

Thus, the term “antibody or antigen-binding fragment thereof”, as used herein, refers to immunoglobulin molecules and immunologically active portions of immunoglobulin molecules, i.e. molecules that contain an antigen-binding site that immunospecifically binds an antigen.

Antibodies and antigen-binding fragments thereof usable in the invention may be from any animal origin including birds and mammals. Preferably, the antibodies or fragments are from human, chimpanzee, rodent (e.g. mouse, rat, guinea pig, or rabbit), chicken, turkey, pig, sheep, goat, camel, cow, horse, donkey, cat, or dog origin. It is particularly preferred that the antibodies are of human or murine origin. Antibodies of the invention also include chimeric molecules in which an antibody constant region derived from one species, preferably human, is combined with the antigen binding site derived from another species, e.g. mouse. Moreover antibodies of the invention include humanized molecules in which the antigen binding sites of an antibody derived from a non-human species (e.g. from mouse) are combined with constant and framework regions of human origin.

As exemplified herein, antibodies of the invention can be obtained directly from hybridomas which express the antibody, or can be cloned and recombinantly expressed in a host cell (e.g., a CHO cell, or a lymphocytic cell). Further examples of host cells are microorganisms, such as E. coli, and fungi, such as yeast. Alternatively, they can be produced recombinantly in a transgenic non-human animal or plant.

The term “chimeric antibody” refers to those antibodies wherein one portion of each of the amino acid sequences of heavy and light chains is homologous to corresponding sequences in antibodies derived from a particular species or belonging to a particular class, while the remaining segment of the chain is homologous to corresponding sequences in another species or class. Typically the variable region of both light and heavy chains mimics the variable regions of antibodies derived from one species of mammals, while the constant portions are homologous to sequences of antibodies derived from another. One clear advantage to such chimeric forms is that the variable region can conveniently be derived from presently known sources using readily available B-cells or hybridomas from non-human host organisms in combination with constant regions derived from, for example, human cell preparations. While the variable region has the advantage of ease of preparation and the specificity is not affected by the source, the constant region being human is less likely to elicit an immune response from a human subject when the antibodies are injected than would the constant region from a non-human source. However, the definition is not limited to this particular example.

The term “humanized antibody” refers to a molecule having an antigen binding site that is substantially derived from an immunoglobulin from a non-human species, wherein the remaining immunoglobulin structure of the molecule is based upon the structure and/or sequence of a human immunoglobulin. The antigen binding site may either comprise complete variable domains fused onto constant domains or only the complementarity determining regions (CDR) grafted onto appropriate framework regions in the variable domains. Antigen-binding sites may be wild-type or modified by one or more amino acid substitutions, e.g. modified to resemble human immunoglobulins more closely. Some forms of humanized antibodies preserve all CDR sequences (for example a humanized mouse antibody which contains all six CDRs from the mouse antibody). Other forms have one or more CDRs which are altered with respect to the original antibody.

Different methods for humanizing antibodies are known to the skilled person, as reviewed by Almagro & Fransson, the content of which is herein incorporated by reference in its entirety (Almagro J C and Fransson J (2008) Frontiers in Bioscience 13:1619-1633). Almagro & Fransson distinguish between rational approaches and empirical approaches. Rational approaches are characterized by generating few variants of the engineered antibody and assessing their binding or any other property of interest. If the designed variants do not produce the expected results, a new cycle of design and binding assessment is initiated. Rational approaches include CDR grafting, Resurfacing, Superhumanization, and Human String Content Optimization. In contrast, empirical approaches are based on the generation of large libraries of humanized variants and selection of the best clones using enrichment technologies or high-throughput screening. Accordingly, empirical approaches are dependent on a reliable selection and/or screening system that is able to search through a vast space of antibody variants. In vitro display technologies, such as phage display and ribosome display fulfill these requirements and are well-known to the skilled person. Empirical approaches include FR libraries, Guided selection, Framework-shuffling, and Humaneering.

The term “human antibody”, as used herein, is intended to include antibodies having variable and constant regions derived from human germline immunoglobulin sequences. The human mAbs of the invention may include amino acid residues not encoded by human germline immunoglobulin sequences (e.g., mutations introduced by random or site-specific mutagenesis in vitro or by somatic mutation in vivo), for example in the CDRs and in particular CDR3. However, the term “human antibody”, as used herein, is not intended to include mAbs in which CDR sequences derived from the germline of another mammalian species (e.g., mouse), have been grafted onto human FR sequences. Human antibodies of the invention include antibodies isolated from human immunoglobulin libraries or from animals transgenic for one or more human immunoglobulin and that do not express endogenous immunoglobulins, as described for example in U.S. Pat. No. 5,939,598 by Kucherlapati & Jakobovits.

The term “monoclonal antibody” as used herein refers to a preparation of antibody molecules of single molecular composition. A monoclonal antibody displays a single binding specificity and affinity for a particular epitope. In one embodiment, the monoclonal antibodies are produced by a hybridoma which includes a B cell obtained from a non-human animal, e.g. mouse, fused to an immortalized cell.

The term “recombinant antibody”, as used herein, includes all antibodies that are prepared, expressed, created or isolated by recombinant means, such as (a) antibodies isolated from an animal (e.g., a mouse) that is transgenic or transchromosomal with respect to the immunoglobulin genes or a hybridoma prepared therefrom, (b) antibodies isolated from a host cell transformed to express the antibody, e.g. from a transfectoma, (c) antibodies isolated from a recombinant, combinatorial antibody library, and (d) antibodies prepared, expressed, created or isolated by any other means that involve splicing of immunoglobulin gene sequences to other DNA sequences.

The term “transfectoma”, as used herein, includes recombinant eukaryotic host cells expressing an antibody, such as CHO cells, NS/0 cells, HEK293 cells, HEK293T cells, plant cells, or fungi, including yeast cells.

As used herein, a “heterologous antibody” is defined in relation to a transgenic organism producing such an antibody. This term refers to an antibody having an amino acid sequence or an encoding nucleic acid sequence corresponding to that found in an organism not consisting of the transgenic organism, and being generally derived from a species other than the transgenic organism.

As used herein, a “heterohybrid antibody” refers to an antibody having light and heavy chains of different organismal origins. For example, an antibody having a human heavy chain associated with a murine light chain is a heterohybrid antibody.

Thus, “antibodies and antigen-binding fragments thereof” suitable for use in the present invention include, but are not limited to, polyclonal, monoclonal, monovalent, bispecific, heteroconjugate, multispecific, recombinant, heterologous, heterohybrid, chimeric, humanized (in particular CDR-grafted), deimmunized, or human antibodies, Fab fragments, Fab′ fragments, F(ab′)₂ fragments, fragments produced by a Fab expression library, Fd, Fv, disulfide-linked Fvs (dsFv), single chain antibodies (e.g. scFv), diabodies or tetrabodies (Holliger P. et al. (1993) Proc. Natl. Acad. Sci. U.S.A. 90(14), 6444-6448), nanobodies (also known as single domain antibodies), anti-idiotypic (anti-Id) antibodies (including, e.g., anti-Id antibodies to antibodies of the invention), and epitope-binding fragments of any of the above.

The antibodies described herein are preferably isolated. An “isolated antibody”, as used herein, is intended to refer to an antibody that is substantially free of other mAbs having different antigenic specificities (e.g., an isolated antibody that specifically binds hPCSK9 is substantially free of mAbs that specifically bind antigens other than hPCSK9). An isolated antibody that specifically binds hPCSK9 may, however, have cross-reactivity to other antigens, such as PCSK9 molecules from other species.

As used herein, a “PCSK9 antagonist” denotes a compound that inhibits at least one biological activity of PCSK9, preferably the proteinase activity of PCSK9. Preferred PCSK9 antagonists are characterized in that they bind from 10% to 100% (preferably from 50% to 100%) of the PCSK9 present in the blood when used in stoichiometric amounts. Preferred PCSK9 antagonists of the present invention are neutralizing antibodies.

A “neutralizing antibody”, as used herein (or an “antibody that neutralizes PCSK9 activity”), is intended to refer to an antibody whose binding to hPCSK9 results in inhibition of at least one biological activity of PCSK9, preferably inhibition of the proteinase activity of PCSK9. This inhibition of the biological activity of PCSK9 can be assessed by measuring one or more indicators of PCSK9 biological activity by one or more of several standard in vitro or in vivo assays known in the art. Such assays are described for example in US 2010/0166768 A1, the content of which is hereby incorporated by reference in its entirety.

Since PCSK9 increases plasma LDL cholesterol by promoting degradation of the LDL receptor, the activity of PCSK9 has an effect on several diseases associated with increased plasma LDL cholesterol levels. Accordingly, PCSK9 antagonists, such as neutralizing anti-hPCSK9 antibodies or antigen-binding fragments thereof, are useful to reduce elevated total cholesterol, non-HDL cholesterol, LDL cholesterol, and/or apolipoprotein B100 (ApoB100). Consequently, PCSK9 antagonists are useful for ameliorating, improving, inhibiting or preventing several such diseases, including without limitation hypercholesterolemia, hyperlipidemia, dyslipidemia, atherosclerosis and cardiovascular diseases.

In specific embodiments, the anti-PCSK9 antibodies or antigen-binding fragments thereof described herein may be conjugated to a therapeutic moiety (“immunoconjugate”), such as a cytotoxin, a chemotherapeutic drug, an immunosuppressant or a radioisotope.

A “conservative amino acid substitution” is one in which an amino acid residue is substituted by another amino acid residue having a side chain (R group) with similar chemical properties (e.g., charge or hydrophobicity). In general, a conservative amino acid substitution will not substantially change the functional properties of a protein. In cases where two or more amino acid sequences differ from each other by conservative substitutions, the percent or degree of similarity may be adjusted upwards to correct for the conservative nature of the substitution. Means for making this adjustment are well known to those of skill in the art. See, e.g., Pearson (1994) Methods MoI. Biol. 24: 307-331. Examples of groups of amino acids that have side chains with similar chemical properties include
1) aliphatic side chains: glycine, alanine, valine, leucine and isoleucine;
2) aliphatic-hydroxyl side chains: serine and threonine;
3) amide-containing side chains: asparagine and glutamine;
4) aromatic side chains: phenylalanine, tyrosine, and tryptophan;
5) basic side chains: lysine, arginine, and histidine;
6) acidic side chains: aspartate and glutamate, and
7) sulfur-containing side chains: cysteine and methionine.

Preferred conservative amino acids substitution groups are: valine-leucine-isoleucine, phenylalanine-tyrosine, lysine-arginine, alanine-valine, glutamate-aspartate, and asparagine-glutamine. Alternatively, a conservative replacement is any change having a positive value in the PAM250 log-likelihood matrix disclosed in Gonnet et al. (1992) Science 256: 1443-45. A “moderately conservative” replacement is any change having a nonnegative value in the PAM250 log-likelihood matrix. Given the known genetic code, and recombinant and synthetic DNA techniques, the skilled scientist can readily construct DNAs encoding conservative amino acid variants.

As used herein, “non-conservative substitutions” or “non-conservative amino acid exchanges” are defined as exchanges of an amino acid by another amino acid listed in a different group of the seven standard amino acid groups 1) to 7) shown above.

The term “substantial identity” or “substantially identical,” when referring to a nucleic acid or fragment thereof, indicates that, when optimally aligned with appropriate nucleotide insertions or deletions with another nucleic acid (or its complementary strand), there is nucleotide sequence identity in at least about 90%, and more preferably at least about 95%, 96%, 97%, 98% or 99% of the nucleotide bases, as measured by any well-known algorithm of sequence identity, such as FASTA, BLAST or GAP, as discussed below.

As applied to polypeptides, the term “substantial similarity” or “substantially similar” means that two peptide sequences, when optimally aligned, such as by the programs GAP or BESTFIT using default gap weights, share at least 90% sequence identity, even more preferably at least 95%, 98% or 99% sequence identity. Preferably, residue positions which are not identical differ by conservative amino acid substitutions.

Sequence similarity for polypeptides is typically measured using sequence analysis software. Protein analysis software matches similar sequences using measures of similarity assigned to various substitutions, deletions and other modifications, including conservative amino acid substitutions. For instance, GCG software contains programs such as GAP and BESTFIT which can be used with default parameters to determine sequence homology or sequence identity between closely related polypeptides, such as homologous polypeptides from different species of organisms or between a wild type protein and a mutein thereof. See, e.g., GCG Version 6.1. Polypeptide sequences also can be compared using FASTA with default or recommended parameters; a program in GCG Version 6.1. FASTA (e.g., FASTA2 and FASTA3) provides alignments and percent sequence identity of the regions of the best overlap between the query and search sequences (Pearson (2000) supra). Another preferred algorithm when comparing a sequence of the invention to a database containing a large number of sequences from different organisms is the computer program BLAST, especially BLASTP or TBLASTN, using default parameters. See, e.g., Altschul et al. (1990) J. Mol. Biol. 215: 403 410 and (1997) Nucleic Acids Res. 25:3389 402, each of which is herein incorporated by reference.

When percentages of sequence identity are referred to in the present application, these percentages are calculated in relation to the full length of the longer sequence, if not specifically indicated otherwise. This calculation in relation to the full length of the longer sequence applies both to nucleic acid sequences and to polypeptide sequences.

As used herein, “treat”, “treating” or “treatment” of a disease or disorder means accomplishing one or more of the following: (a) reducing the severity and/or duration of the disorder; (b) limiting or preventing development of symptoms characteristic of the disorder(s) being treated; (c) inhibiting worsening of symptoms characteristic of the disorder(s) being treated; (d) limiting or preventing recurrence of the disorder(s) in patients that have previously had the disorder(s); and (e) limiting or preventing recurrence of symptoms in patients that were previously symptomatic for the disorder(s).

As used herein, “prevent”, “preventing”, “prevention”, or “prophylaxis” of a disease or disorder means preventing that a disorder occurs in subject.

As used herein, the expressions “is for administration” and “is to be administered” have the same meaning as “is prepared to be administered”. In other words, the statement that an active compound “is for administration” has to be understood in that said active compound has been formulated and made up into doses so that said active compound is in a state capable of exerting its therapeutic activity.

The terms “therapeutically effective amount” or “therapeutic amount” are intended to mean that amount of a drug or pharmaceutical agent that will elicit the biological or medical response of a tissue, a system, animal or human that is being sought by a researcher, veterinarian, medical doctor or other clinician. The term “prophylactically effective amount” is intended to mean that amount of a pharmaceutical drug that will prevent or reduce the risk of occurrence of the biological or medical event that is sought to be prevented in a tissue, a system, animal or human by a researcher, veterinarian, medical doctor or other clinician. Particularly, the dosage a patient receives can be selected so as to achieve the amount of LDL (low density lipoprotein) cholesterol lowering desired; the dosage a patient receives may also be titrated over time in order to reach a target LDL level. The dosage regimen utilizing an antibody or an antigen-binding fragment thereof as described herein is selected in accordance with a variety of factors including type, species, age, weight, body mass index, sex and medical condition of the patient; the severity of the condition to be treated; the potency of the compound chosen to be administered; the route of administration; the purpose of the administration; and the renal and hepatic function of the patient.

As used herein, a “patient” means any human or non-human animal, such as mammal, reptile or bird who may benefit from a treatment with the antibodies and antigen-biding fragments thereof described herein. Preferably, a “patient” is selected from the group consisting of laboratory animals (e.g. mouse or rat), domestic animals (including e.g. guinea pig, rabbit, chicken, turkey, pig, sheep, goat, camel, cow, horse, donkey, cat, or dog), rodent or primates including chimpanzee, gorilla, bonobo and human beings. It is particularly preferred that the “patient” is a human being.

The terms “subject” or “individual” are used interchangeably herein. As used herein, a “subject” refers to a human or a non-human animal (e.g. a mammal, avian, reptile, fish, amphibian or invertebrate; preferably an individual that can either benefit from one of the different aspects of present invention (e.g. a method of treatment or a drug identified by present methods) or that can be used as laboratory animal for the identification or characterisation of a drug or a method of treatment. The individual can e.g. be a human, a wild-animal, domestic animal or laboratory animal; examples comprise: mammal, e.g. human, non-human primate (chimpanzee, bonobo, gorilla), dog, cat, rodent (e.g. mouse, guinea pig, rat, hamster or rabbit, horse, donkey, cow, sheep, goat, pig, camel; avian, such as duck, dove, turkey, goose or chick; reptile such as: turtle, tortoise, snake, lizard, amphibian such as frog (e.g. Xenopus laevis); fish such as koy or zebrafish; invertebrate such as a worm (e.g. c. elegans) or an insect (such as a fly, e.g. drosophila melanogaster). The term individual also comprises the different morphological developmental stages of avian, fish, reptile or insects, such as egg, pupa, larva or imago. It is further preferred if the subject is a “patient”.

As used herein, “unit dosage form” refers to physically discrete units suitable as unitary dosages for human and/or animal subjects, each unit containing a predetermined quantity of active material (e.g., about 50 to about 500 mg of PCSK5 antibody and/or of e.g. 0.05 mg to 100 mg HMG-CoA reductase inhibitor) calculated to produce the desired therapeutic effect in association with the required pharmaceutical diluent, carrier or vehicle. The specifications for the novel unit dosage forms of this invention are dictated by and are directly dependent on (a) the unique characteristics of the active material and the particular therapeutic effect to be achieved, and (b) the limitation inherent in the art of compounding such an active material for therapeutic use in animals or humans, as disclosed in this specification, these being features of the present invention. Examples of suitable unit dosage forms in accord with this invention are vials, tablets, capsules, troches, suppositories, powder packets, wafers, cachets, ampules, segregated multiples of any of the foregoing, and other forms as herein described or generally known in the art. One or more such unit dosage forms of the antibody can be comprised in an article of manufacture of present invention, optionally further comprising one or more unit dosage forms of an HMG-CoA reductase inhibitor (e.g. a blister of tablets comprising as active ingredient the HMG-CoA reductase inhibitor).

The term “active material” refers to any material with therapeutic activity, such as one or more active ingredients. The active ingredients to be employed as therapeutic agents can be easily prepared in such unit dosage form with the employment of pharmaceutical materials which themselves are available in the art and can be prepared by established procedures.

The following preparations are illustrative of the preparation of the active ingredients and unit dosage forms of the present invention, and not as a limitation thereof. Several dosage forms may be prepared embodying the present invention. For example, a unit dosage per vial may contain 0.5 ml, 1 ml, 2 ml, 3 ml, 4 ml, 5 ml, 6 ml, 7 ml, 8 ml, 9 ml, 10 ml, 15 ml, or 20 ml of PCSK5 antibody or a fragment thereof ranging from about 40 to about 500 mg of PCSK5 antibody. If necessary, these preparations can be adjusted to a desired concentration by adding a sterile diluent to each vial. In one embodiment, the ingredients of formulation of the invention are supplied either separately or mixed together in unit dosage form, for example, as a dry lyophilized powder or water free concentrate in a hermetically sealed container such as a vial, an ampoule or sachette indicating the quantity of active agent. Where the composition is to be administered by infusion, it can be dispensed with an infusion bottle containing sterile pharmaceutical grade water or saline. Where the composition is administered by injection, an ampoule of sterile water for injection or saline can be provided so that the ingredients may be mixed prior to administration.

The formulations of the invention include bulk drug compositions useful in the manufacture of pharmaceutical compositions (e.g., compositions that are suitable for administration to a subject or patient) which can be used in the preparation of unit dosage forms. In a preferred embodiment, a composition of the invention is a pharmaceutical composition. Such compositions comprise a prophylactically or therapeutically effective amount of one or more prophylactic or therapeutic agents (e.g., an antibody of the invention or other prophylactic or therapeutic agent), and a pharmaceutically acceptable carrier. Preferably, the pharmaceutical compositions are formulated to be suitable for the route of administration to a subject.

The active materials or ingredients (e.g. antibodies or fragments thereof and HMG-CoA reductase inhibitors) can be formulated as various dosage forms including solid dosage forms for oral administration such as capsules, tablets, pills, powders and granules, liquid dosage forms for oral administration such as pharmaceutically acceptable emulsions, microemulsions, solutions, suspensions, syrups and elixirs, injectable preparations, for example, sterile injectable aqueous or oleaginous suspensions, compositions for rectal or vaginal administration, preferably suppositories, and dosage forms for topical or transdermal administration such as ointments, pastes, creams, lotions, gels, powders, solutions, sprays, inhalants or patches.

In a specific embodiment, the term “pharmaceutically acceptable” means approved by a regulatory agency of the U.S. Federal or a state government or the EMA (European Medicines Agency) or listed in the U.S. Pharmacopeia Pharmacopeia (United States Pharmacopeia-33/National Formulary-28 Reissue, published by the United States Pharmacopeial Convention, Inc., Rockville Md., publication date: April 2010) or other generally recognized pharmacopeia for use in animals, and more particularly in humans. The term “carrier” refers to a diluent, adjuvant {e.g., Freund's adjuvant (complete and incomplete)), excipient, or vehicle with which the therapeutic is administered. Such pharmaceutical carriers can be sterile liquids, such as water and oils, including those of petroleum, animal, vegetable or synthetic origin, such as peanut oil, soybean oil, mineral oil, sesame oil and the like. Water is a preferred carrier when the pharmaceutical composition is administered intravenously. Saline solutions and aqueous dextrose and glycerol solutions can also be employed as liquid carriers, particularly for injectable solutions. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. For the use of (further) excipients and their use see also “Handbook of Pharmaceutical Excipients”, fifth edition, R. C. Rowe, P. J. Seskey and S. C. Owen, Pharmaceutical Press, London, Chicago. The composition, if desired, can also contain minor amounts of wetting or emulsifying agents, or pH buffering agents. These compositions can take the form of solutions, suspensions, emulsion, tablets, pills, capsules, powders, sustained-release formulations and the like. Oral formulation can include standard carriers such as pharmaceutical grades of mannitol, lactose, starch, magnesium stearate, sodium saccharine, cellulose, magnesium carbonate, etc. Examples of suitable pharmaceutical carriers are described in “Remington's Pharmaceutical Sciences” by E.W. Martin. Such compositions will contain a prophylactically or therapeutically effective amount of the antibody, preferably in purified form, together with a suitable amount of carrier so as to provide the form for proper administration to the patient. The formulation should suit the mode of administration.

Generally, the ingredients of compositions of the invention are supplied either separately or mixed together in unit dosage form, for example, as a dry formulation for dissolution such as a lyophilized powder, freeze-dried powder or water free concentrate in a hermetically sealed container such as an ampoule or sachette indicating the quantity of active agent. The ingredients of compositions of the invention can also be supplied as admixed liquid formulation (i.e. injection or infusion solution) in a hermetically sealed container such as an ampoule, sachette, a pre-filled syringe or autoinjector, or a cartridge for a reusable syringe or applicator (e.g. pen or autoinjector). Where the composition is to be administered by infusion, it can be dispensed with an infusion bottle containing sterile pharmaceutical grade water or saline. Where the composition is administered by injection, an ampoule of sterile water for injection or saline can be provided so that the ingredients may be mixed prior to administration.

The invention also provides that the formulation is packaged in a hermetically sealed container such as an ampoule or sachette indicating the quantity of antibody. In one embodiment, the formulation of the invention comprising an antibody is supplied as a dry formulation, such as a sterilized lyophilized powder, freeze-dried powder, spray-dried powder or water free concentrate in a hermetically sealed container and can be reconstituted, e.g., with water or saline to the appropriate concentration for administration to a subject. In another embodiment the antibody or antigen binding fragment thereof is supplied as a liquid formulation such as an injection or infusion solution. In one embodiment, the formulation of the invention comprising an antibody is supplied as a dry formulation or as a liquid formulation in a hermetically sealed container at a unit dosage of at least 40 mg, at least 50 mg, more preferably at least 75 mg, at least 100 mg, at least 150 mg, at least 200 mg, at least 250 mg, at least 300 mg, at least 350 mg, at least 400 mg, at least 450 mg, or at least 500 mg, of antibody or antigen-binding fragment thereof. The lyophilized formulation of the invention comprising an antibody should be stored at between 2 and 8° C. in its original container and the antibody should be administered within 12 hours, preferably within 6 hours, within 5 hours, within 3 hours, or within 1 hour after being reconstituted. The formulation of the invention comprising antibodies can be formulated as neutral or salt forms. Pharmaceutically acceptable salts include those formed with anions such as those derived from hydrochloric, phosphoric, acetic, oxalic, tartaric acids, etc., and those formed with cations such as those derived from sodium, potassium, ammonium, calcium, ferric hydroxides, isopropylamine, triethylamine, 2-ethylamino ethanol, histidine, procaine, etc.

Adult subjects are characterized as having “hypertension” or a high blood pressure when they have a systolic blood pressure of more than 140 mmHg and/or a diastolic blood pressure of more than 90 mmHg.

Specific populations treatable by the therapeutic methods of the invention include subjects with one or more of the following conditions: subjects indicated for LDL apheresis, subjects with PCSK9-activating mutations (gain of function mutations, “GOF”), subjects with elevated total cholesterol levels, subjects with elevated low-density lipoprotein cholesterol (LDL-C) levels, subjects with primary hypercholesterolemia, such as subjects primary with Familial or Non-Familial Hypercholesterolemia, subjects with heterozygous Familial Hypercholesterolemia (heFH); subjects with hypercholesterolemia, especially primary hypercholesterolemia, who are statin intolerant or statin uncontrolled; and subjects at risk for developing hypercholesterolemia who may be preventably treated. Other indications include hyperlipidemia and dyslipidemia, especially if associated with secondary causes such as Type 2 diabetes mellitus, cholestatic liver diseases (primary biliary cirrhosis), nephrotic syndrome, hypothyroidism, obesity; and the prevention and treatment of atherosclerosis and cardiovascular diseases, such as coronary heart disease (CHD). The conditions or disorders as listed for the above populations or subjects are conditions or disorders, for which treatment with the antibody of the invention is especially suitable.

However, depending on the severity of the afore-mentioned diseases and conditions, the treatment of subjects with the antibodies and antigen-binding fragments of the invention may be contraindicated for certain diseases and conditions.

The term “adverse effect” (or side-effect) refers to a harmful and undesired effect resulting from a medication. An adverse effect may be termed a “side effect”, when judged to be secondary to a main or therapeutic effect. Some adverse effects occur only when starting, increasing or discontinuing a treatment. Adverse effects may cause medical complications of a disease and negatively affect its prognosis. Examples of side effects are allergic reactions, vomiting, headache, or dizziness or any other effect herein described.

As used herein, “treat”, “treating” or “treatment” of a disease or disorder means accomplishing one or more of the following: (a) reducing the severity and/or duration of the disorder; (b) limiting or preventing development of symptoms characteristic of the disorder(s) being treated; (c) inhibiting worsening of symptoms characteristic of the disorder(s) being treated; (d) limiting or preventing recurrence of the disorder(s) in patients that have previously had the disorder(s); and (e) limiting or preventing recurrence of symptoms in patients that were previously symptomatic for the disorder(s).

As used herein, “prevent”, “preventing”, “prevention”, or “prophylaxis” of a disease, condition or disorder means preventing that a disorder, disease or condition occurs in subject. Elevated total cholesterol levels of human beings are understood in the context of present invention to be total cholesterol levels of 200 mg/dL or more, especially 240 mg/dL or more, elevated LDL-C levels are understood in the context of present invention to be LDL-C levels of 100 mg/dL or more, preferably mg/dL or more, preferably 160 mg/dL or more such as 190 mg/dL or more Low High-density lipoprotein levels (HDL-levels) in the context of present invention are understood to be less than about 40 mg/dL.

The terms “sample” or “taken sample” are used synonymously herein and are understood in the context of the different aspects of present invention, to preferably refer to a biologically sample. The term “sample” or “sample of interest” are used interchangeably herein, referring to a small part intended to represent the whole of a tissue, an organ or an individual. Upon analysis a sample provides information about the tissue status or the health or diseased status of an organ or individual. Examples of samples include but are not limited to fluid samples such as cerebrospinal fluid, blood, serum, plasma, synovial fluid, urine, saliva, and lymphatic fluid, or solid samples such as biopsy samples, tissue, and tissue-extracts, e.g. taken from nervous tissue (e.g from the spinal cord), skin, muscle, cartilage, bone, synovium, perichondrium, capsule, and connective tissue. Further examples of samples are cell cultures or tissue cultures such as but not limited to cultures of neural cells.

Analysis of a sample may be accomplished on a visual or chemical basis. Visual analysis includes but is not limited to microscopic imaging or radiographic scanning of a tissue, organ or individual allowing for morphological evaluation of a sample. Chemical analysis includes but is not limited to the detection of the presence or absence of specific indicators or alterations in their amount or level. For example, a tissue sample may be removed from a subject by conventional biopsy techniques or a blood sample may be taken from a subject by conventional blood collection techniques. The sample, e.g. tissue or blood sample, may be obtained from a subject prior to initiation of the therapeutic treatment, during the therapeutic treatment, and/or after the therapeutic treatment, e.g. with a pain reducing compound.

It is preferred that the sample is a body fluid sample, a tissue sample, a cell colony sample, a single cell sample or a cell culture sample. More preferably, the tissue sample is a section or an explant sample, e.g. an explant sample of dorsal root ganglia or spinal cord. The term “body fluid sample” refers to a liquid sample derived from the body of a subject. Said body fluid sample may be a blood, urine, cerebrospinal fluid, cerumen (earwax), endolymph, perilymph, gastric juice, mucus, peritoneal fluid, pleural fluid, saliva, or sebum (skin oil) sample including components or fractions thereof. Said body fluid samples may be mixed or pooled. Thus, a body fluid sample may be a mixture of a blood sample and anurine sample or a mixture of a blood sample and cerebrospinal fluid sample. A “body fluid sample” may be provided by removing a body liquid from a subject, but may also be provided by using previously isolated body fluid sample material. Preferably, the blood sample of a subject is whole blood or a blood fraction such as serum or plasma. It is also particularly preferred to use blood cells also known as hemopoietic cells.

It is preferred that the tissue sample has a weight of between 0.1 and 500 mg, more preferably of between 0.5 and 250 mg, and most preferably of between 1 and 50 mg, i.e. 0.1, 0.2, 0.3, 0.4, 0.5, 0.6, 0.7, 0.8, 0.9, 1, 2, 2.5, 3, 4, 5, 6, 7, 8, 9, 10, 15, 20, 25, 30, 35, 40, 45, 50, 55, 60, 65, 70, 75, 80, 85, 90, 95, 100, 125, 150, 175, 200, 225, 250, 275, 300, 325, 350, 375, 400, 425, 450, 475, or 500 mg.

It is also preferred that the cell sample (e.g. cell colony sample or cell culture sample) consists of between 100 and 1000 cells, more preferably of between 200 and 800 cells, and most preferably of between 400 and 600 cells.

It is further preferred that the body fluid sample has a volume of between 0.1 and 20 ml, more preferably of between 0.5 and 10 ml, more preferably between 1 and 8 ml and most preferably between 2 and 5 ml, i.e. 0.1, 0.2, 0.3, 0.4, 0.5, 0.6, 0.7, 0.8, 0.9, 1, 2, 2.5, 3, 4, 5, 6, 7, 8, 9, 10, 11, 12, 13, 14, 15, 16, 17, 18, 19, or 20 ml. More preferably, the blood sample has a volume of between 0.1 and 20 ml, more preferably of between 0.5 and 10 ml, and most preferably of between 1 and 5 ml, i.e. 0.1, 0.2, 0.3, 0.4, 0.5, 0.6, 0.7, 0.8, 0.9, 1, 2, 2.5, 3, 4, 5, 6, 7, 8, 9, 10, 11, 12, 13, 14, 15, 16, 17, 18, 19, or 20 ml.

In the context of the present invention, the term “kit of parts” (in short: “kit”) is understood to be an article of manufacture comprising a combination of two or more of the components identified in this application, which are combined, coexisting spatially, to a functional unit, and which can contain further components.

In the context of the present invention, the term “significant effect” means any effect different from the background noise of a(ny) experiment. If the test compound has a significant effect on the test system, the test compound is identified as compound that e.g. modulates cholesterol levels. For this, the effect of the test compound is compared to a control, particularly a negative control. A (statistically) significant effect in drug screening is e.g. an effect that is higher or lower than the mean value of the experiment and at the same time also higher or lower than the standard deviation (e.g. higher or lower than the mean value of an experiment (with double or triple determination of values in an experiment) plus/minus the 3-fold Standard Deviation (mean±3SD)). The skilled person knows how to discriminate significant effects from background noise or experimental deviations.

The term “antagonist” as used herein refers to a substance blocking the action of an agonist. Typically, antagonists act by binding to the active site or to allosteric sites of a receptor molecule, or interact with unique binding sites not normally involved in the regulation of the activity of the receptor. Typically, an antagonist competes with the agonist at structurally-defined binding sites. The antagonist activity may be reversible or irreversible depending on the longevity of the interaction of the antagonist-receptor complex. Examples for antagonists include but are not limited to nucleic acid molecules, such as siRNAs or miRNAs, or proteins such as hormones, cytokines, growth factors or neurotransmitter, antibodies, or transcription factors.

### EMBODIMENTS OF THE INVENTION

The present invention will now be further described. In the following passages different aspects of the invention are defined in more detail. Each aspect so defined may be combined with any other aspect or aspects unless clearly indicated to the contrary. In particular, any feature indicated as being preferred or advantageous may be combined with any other feature or features indicated as being preferred or advantageous, unless clearly indicated to the contrary.

In a first aspect, present invention is directed to method for screening compounds to identify therapeutic candidates for the modulation of a disease or condition associated with elevated LDL-C levels, said method comprising:
(a) providing a rodent
(b) administering a test compound to the rodent and
(c) detecting whether said compound increases or decreases one or more parameters selected from the group consisting of: total cholesterol (TC), low-density cholesterol (LDL-C) and high-density cholesterol (HDL-C) in said rodent in comparison to a control rodent;
   wherein a modulation of one or more of said parameters indicates that said compound is a candidate for modulating said disease or condition in vivo.

In this aspect, the control rodent is a different animal, i.e. a different individual, than the rodent. It is also possible to determine the cholesterol level in two or more control animals and to calculate the mean value of the cholesterol level in these two or more control animals.

In a second aspect, present invention is directed to a method for screening compounds to identify therapeutic candidates for the modulation of a disease or condition associated with elevated LDL-C level, said method comprising:
(a) providing a rodent
(b) administering a test compound to the rodent
(c) determining one or more parameters of the rodent selected from the group consisting of: total cholesterol (TC), low-density cholesterol (LDL-C) and high-density cholesterol (HDL-C) before treatment of the rodent with the compound
(d) determining the one or more parameters after treatment of the rodent with the compound, and
(e) comparing the results obtained in (a) with those obtained in (b),
   wherein a difference of the parameters of (a) in comparison with those of (b) indicates that the compound is a candidate for modulating said disease or condition in vivo.

In this aspect, the cholesterol level in the rodent after administration of the test compound is compared to a pre-dose cholesterol level of cholesterol in the same animal.

According to a preferred embodiment of the first and second aspect, the parameters are determined in vitro in one or more taken samples of the rodent or rodents.

The determination of the parameters or the taking of the samples that occur after a compound (e.g. antibody or statin) has been administered to the rodent, are preferably timed in such a manner that the determination or the sample taking takes place after a time sufficient for the compound to exert its effect.

In aspects or embodiments, which employ control rodents or samples obtained therefrom as comparison, it is suitable if the control rodents obtain placebos preferably comprising the same ingredients as the administered compound-containing formulation apart from the active ingredient itself. Moreover, it is suitable if the placebo administration and the sample-taking and/or parameter-determination occurs at the same time or with the same timing as for the test-rodent receiving the compound. The administered placebo moreover preferably has the same formulation (liquid or solid, buffer composition) and is administered via the same route (e.g. peroral, oral, topical or per injection) and with the same unit dosage form (e.g. a liquid of a certain volume or a tablet etc. . . . ).

Another preferred embodiment of the invention concerns an in vitro method based on step (c) of the method according to the first aspect of based on steps (c), (d) and (e) of the second aspect.

According to another preferred embodiment of the first two aspects, the modulation of the one or more parameters is indicative of the same in vivo effect in other mammals such as humans, or in reptiles or birds.

According to another preferred embodiment of the first two aspects,
a decrease of total cholesterol and/or of LDL-C and/or increase of HDL-C is indicative that said compound is a candidate for treating or preventing one or more of said diseases or conditions in vivo, and wherein
an increase of total cholesterol and/or of LDL-C is indicative that said compound exhibits adverse effects and is a candidate for promoting or inducing one or more of said diseases or conditions in vivo.

As lowered LDL-C levels and total cholesterol levels and increases HDL-C levels are connected with decreased risks for developing diseases or conditions connected therewith (e.g. hyperlipidemia, cardiovascular diseases etc), the lowering of said parameters in the test rodent is indicative that the tested compound will act as a medicament for the treatment or prevention of one or more such diseases.

As increased total C and LDL-C levels are connected with the onset of these disorders, present methods allow for an identification of adverse effects in the compounds tested. This way, present methods and animal models are also suitable for testing whether candidate compounds intended for the treatment of different diseases than those connected with elevated LDL-C levels are tested for possible adverse effects on the lipid metabolism. Although lowered HDL-C levels may be one such further parameter, this parameter appears not to be indicative in the case of present rodent test model (see example section).

The compound used in the different aspects of present invention can be any active material such as any biological or chemical substance or natural product extract, either purified, partially purified, synthesized or manufactured by means of biochemical or molecular biological methods e.g. a biological molecule, such as a protein [e.g. antibody, a non-antibody protein scaffold (e.g. a darpin, an anticalin, a diabody, an affibody etc.)] a nucleic acid [e.g. antisense, siRNA or aptamer] or a small molecule. According to one embodiment, the compound is an HMG-CoA reductase inhibitor, e.g. a statin.

According to a preferred embodiment of the different aspects of present invention, the HMG-CoA reductase inhibitor is a statin. More preferably, the statin is selected from the group consisting of cerivastatin, atorvastatin, simvastatin, pitavastatin, rosuvastatin, fluvastatin, lovastatin, and pravastatin.

The compound can be the active ingredient alone or contained in a formulation or pharmaceutical composition or admixture, optionally comprising or combined with one or more further active ingredients.

The compound can be administered in any dosage and application regime and in any route of administration suitable for use in rodents. The route of administration will also always depend on the compound that is to be administered and its preferred formulation (e.g. liquid or solid).

Preferred administration regimes according to present invention are once per day, twice per day or three times per day, daily, every other day, once a week, every other week or once a month. Suitable routes of administration comprise e.g. oral administration, peroral administration, topical administration, injection (e.g. intravenous, intramuscular, intraperitoneal or subcutaneous) or infusion.

Suitable dosages of antibody for a rodent are e.g. an amount of about 0.5 mg/kg body weight to about 15 mg/kg body weight and preferably in a concentration of about 0.8 mg/kg body weight to about 13 mg/kg body weight and preferably in a concentration of about, 0.5 mg/kg, about 1 mg/kg body weight, about 2 mg/kg, about 3 mg/kg, about 4 mg/kg, about 5 mg/kg, about 6 mg/kg, about 7 mg/kg about 8 mg/kg about 9 mg/kg, 10 about mg/kg, about 11 mg/kg, about 12 mg/kg or about 13 mg/kg body weight. According to a further preferred embodiment, the antibody is administered to the rodent, preferably a hamster, in an amount of about 1 mg/kg body weight, about 3 mg/kg body weight or about 10 mg/kg body weight.

Preferably such a dosage of antibody is administered to the rodent once, once a week, every other week or once a month. A preferable route of administration for an antibody is e.g. a subcutaneous injection.

According to one embodiment of the first two or the other aspects of present invention, the rodent has has decreased PCSK9 levels or decreased PCSK9 activity in comparison to a reference, e.g. in comparison to a rodent with average PCSK9 levels representative for rodents of the same species and/or the same health condition and/or the same strain and/or the same gender. E.g. if the rodent is a hamster, e.g. a syrian hamster such as a male syrian hamster, the rodent with decreased PCSK9 levels or activity has a PCSK9 level (preferably protein level) or activity (e.g. protein activity) that is significantly lower (e.g. at least 5%, 10%, 15%, 20%, 25% or 30% lower) than that of average hamsters, preferably average syrian hamsters, or average male hamsters or average male syrian hamsters or average hamsters of the same health status (e.g. normolipidemic or healthy) or average hamsters of about the same age, e.g. average male syrian hamsters.

According to another embodiment, present invention relates to a method of aspect 1 or 2, wherein an antagonist of PCSK9 and preferably an antibody specifically binding to PCSK9 has been administered to the rodents of aspect 1 prior to step (c) or to the rodent of aspect 2 prior to step (a). Preferably, the same concentration (measured in mg/kg body weight) of the antibody or antigen-binding fragment thereof is administered to the rodent and to the control animal in aspect one. Likewise, it is possible to challenge two or more rodents with a PCSK-9 antagonist, such as a PCSK-9 antibody or antigen-binding fragment thereof, to determine the cholesterol level in these two or more rodents and to calculate the mean value of the cholesterol level in these two or more rodents.

According to a preferred embodiment of the different aspects of present invention, the compound is a lipid lowering compound, such as a compound lowering LDL-C levels, e.g. an inhibitor of HMG-CoA reductase and preferably a statin, wherein the statin is preferably selected from the group consisting of: cerivastatin, atorvastatin, simvastatin, pitavastatin, rosuvastatin, fluvastatin, lovastatin or pravastatin.

If the compound is an HMG-CoA reductase inhibitor, such as a statin, preferable daily dosages are about 10 mg/kg to about 60 mg/kg, preferably about 15 mg/kg to about 50 mg/kg daily dosage and more preferably about 20 mg/kg to about 40 mg/kg daily dosage, e.g. about 10 mg/kg daily dosage, about 20 mg/kg daily dosage, about 30 mg/kg daily dosage, about 40 mg/kg, about 50 mg/kg or about 60 mg/kg daily dosage. In a further preferred embodiment, the daily dosage of HMG-CoA reductase inhibitor, e.g. of the statin, is administered in a twice per day (or two-times daily) administration regime, e.g. about 5 mg/kg to about 30 mg/kg per single dose twice per day (i.e. about 10 mg/kg to about 60 mg/kg daily dosage), or about 10 mg/kg to about 20 mg/kg per single dose twice per day (i.e. about 20 mg/kg to about 40 mg/kg daily dosage) and preferably about 10 mg/kg twice per day or 15 mg/kg twice per day or 20 mg/kg twice per day or 25 mg/kg twice per day and more preferably about 10 mg/kg twice per day or about 20 mg/kg twice per day.

In further preferred embodiment of the different aspects of present invention, the statin is:
cerivastatin administered in a daily dosage of between 0.05 mg and 2 mg, preferably in a daily dosage of 0.2 mg, 0.4 mg, or 0.8 mg;
atorvastatin administered in a daily dosage of between 2 mg and 100 mg, preferably in a daily dosage of 10 mg, 20 mg, 40 mg, or 80 mg;
simvastatin administered in a daily dosage of between 2 mg and 100 mg, preferably in a daily dosage of 5 mg, 10 mg, 20 mg, 40 mg, or 80 mg;
pitavastatin administered in a daily dosage of between 0.2 mg and 100 mg, preferably in a daily dosage of 1 mg, 2 mg, 5 mg, 10 mg, or 20 mg;
rosuvastatin administered in a daily dosage of between 2 mg and 100 mg, preferably in a daily dosage of 5 mg, 10 mg, 20 mg, or 40 mg;
fluvastatin administered in a daily dosage of between 2 mg and 100 mg, preferably in a daily dosage of 20 mg, 40 mg, or 80 mg;
lovastatin administered in a daily dosage of between 2 mg and 100 mg, preferably in a daily dosage of 10 mg, 20 mg, 40 mg, or 80 mg; or
pravastatin administered in a daily dosage of between 2 mg and 100 mg, preferably in a daily dosage of 10 mg, 20 mg, 40 mg, or 80 mg.

According to another preferred embodiment, the daily dose of statin is administered to the rodent as a twice-a-day administration (i.e. two administrations per day each comprising a part (e.g. half) of the daily dosage and together comprising the full daily dosage.

In a third aspect present invention concerns a method of testing the efficacy of an antibody or an antigen-binding fragment thereof which specifically binds to hPCSK9 for the treatment of a disease or condition associated with elevated LDL-C levels, said method comprising:
(a) administering said antibody to a rodent; and
(b) determining the total cholesterol, LDL-C or HDL-C level of the rodent before and after administration of said antibody or antigen-binding fragment thereof to the rodent,
   wherein a reduction of the total cholesterol and/or LDL-C level and/or a increase of the HDL-C level determined after administration of the antibody relative to the predose level determined before administration of the antibody is indicative that the antibody or antigen-binding fragment thereof is efficacious for the treatment of said disease or condition, and
   wherein the increase of the total cholesterol level and/or the LDL-C level determined after administration of the antibody relative to the predose level determined before administration of the antibody is indicative that the antibody exhibits adverse effects in promoting, contributing to or triggering said disease or condition in vivo.

In a fourth aspect, present invention concerns a method of testing the efficacy of an antibody or an antigen-binding fragment thereof which specifically binds hPCSK9 for the modulation of a disease or condition associated with elevated LDL-C levels, said method comprising:
(a) determining the total cholesterol level, the LDL-C level and/or the HDL-level in an in vitro sample obtained from a rodent before treatment of the rodent with the antibody,
(b) determining the total cholesterol level, the LDL-C level and/or the HDL-level in an in vitro sample obtained from the rodent after treatment of the rodent with the antibody, and
(c) comparing the results obtained in (a) with those obtained in (b),
   wherein a reduction of the total cholesterol and/or LDL-C level and/or a increase of the HDL-C level determined in (b) relative to the predose level determined in (a) before administration of the antibody is indicative that the antibody or antigen-binding fragment thereof is efficacious for the treatment and/or prevention of said disease or condition, and
   wherein the increase of the total cholesterol level and/or the LDL-C level in (b) in comparison the predose level detected in (a) is indicative that the antibody exhibits adverse effects in promoting, contributing to or triggering said disease or condition in vivo.

According to a preferred embodiment of the third or fourth aspect, the rodent has been administered a compound lowering total cholesterol and/or LDL-C levels and/or increasing HDL-C levels in humans and wherein the compound has been administered prior to determining of the predose level in the method of aspect 3 and prior to the taking of the sample in step (a) of aspect 4. According to one embodiment the compound is a statin, e.g atorvastatin.

In a fifth aspect, present invention concerns a method of testing the efficacy of an antibody or an antigen-binding fragment thereof which specifically binds hPCSK9 for the treatment of a disease or condition associated with elevated LDL-C levels, said method comprising:
(a) administering said antibody to a rodent; and
(b) determining the efficacy of said antibody or antigen-binding fragment thereof by determining the total cholesterol level and/or LDL-C level and/or HDL-C level of the rodent after administration of said antibody or antigen-binding fragment thereof,
(c) determining the total cholesterol level and/or LDL-C level and/or HDL-C level of a control rodent that has not been treated with the antibody and has preferably obtained a placebo,
   wherein the antibody is considered efficacious for the treatment of the disease or condition if the total cholesterol level and/or LDL-C level determined in (c) is lower and/or the HDL-C level determined in (c) is higher than that determined in (b) and
   wherein the antibody is considered to exhibit adverse effects if the total cholesterol level and/or LDL-C level determined in (c) is higher than that determined in (b).

In a sixth aspect, present invention concerns an in vitro method of testing the efficacy of an antibody or an antigen-binding fragment thereof which specifically binds hPCSK9 for the treatment of a disease or condition associated with elevated LDL-C levels, said method comprising:
(a) determining the total cholesterol level and/or LDL-C level and/or HDL-C level in a sample of a rodent obtained after administration of said antibody or antigen-binding fragment thereof to the rodent,
(b) determining the total cholesterol level and/or LDL-C level and/or HDL-C level in a control sample obtained from a rodent that has not been treated with said antibody or antigen-binding fragment thereof,
   wherein the antibody is considered efficacious for the treatment of the disease or condition if the total cholesterol level and/or LDL-C level determined in (b) is lower and/or the HDL-C level determined in (b) is higher than that determined in (a) and
   wherein the antibody is considered to exhibit adverse effects if the total cholesterol level and/or LDL-C level determined in (b) is higher than that determined in (a).

According to a preferred embodiment of the fifth or sixth aspect, the rodent and the control rodent have been administered a compound lowering total cholesterol and/or LDL-C and/or increase HDL-C in humans.

According to another preferred embodiment of the fifth or sixth aspect, the compound is an HMG-CoA reductase inhibitor and preferably a statin.

In a seventh aspect, present invention concerns a method for testing the efficacy of a compound in modulating cholesterol levels in a subject, comprising the steps:
(a) providing a rodent;
(b) administering an antibody or an antigen-binding fragment thereof which specifically binds PCSK9 to the rodent;
(c) administering a test compound to said rodent;
(d) determining one or more parameters of the rodent selected from the group consisting of: the total cholesterol level, LDL-C level or HDL-C level, after administration of the test compound,
(e) determining the same one or more parameter(s) of a control rodent that has not been challenged with the test compound
   wherein a difference in the cholesterol (total or LDL-C or HDL-C) determined in (a) and determined in (b) indicates that the test compound is efficacious in modulating cholesterol levels in a subject.

In an eighth aspect, present invention concerns an in vitro method for testing the efficacy of a compound in modulating cholesterol levels in a subject, comprising the steps:
(a) determining in a sample of a rodent taken after the rodent has been applied a test compound one or more of the parameters selected from the group consisting of: the total cholesterol level, LDL-C level or HDL-C level,
(b) determining the same one or more parameter(s) in a sample of a control rodent that has not been challenged with the test compound
   wherein both animals have been administered an antibody or an antigen-binding fragment thereof which specifically binds PCSK9 in addition to the test compound, and
   wherein a difference in the cholesterol (total C and/or LDL-C and/or HDL-C) determined in (a) and determined in (b) indicates that the test compound is efficacious in modulating cholesterol levels in a subject.

According to a preferred embodiment of the seventh or eights aspect of present invention,

a decreased level of cholesterol (total and/or LDL-C) and/or an increased level of HDL-C determined in the rodent or in a sample thereof as compared to the total, LDL or HDL cholesterol level in the control rodent indicates that the test compound is efficacious in the treatment or prevention of one or more of the diseases or disorders associated with elevated LDL-C levels in a subject, and
an increased level of cholesterol (total and/or LDL-C) determined in the test rodent or in a sample thereof as compared to the total- or LDL cholesterol level in the control rodent indicates that the test compound has adverse effects and may promote, contribute to or trigger a disease or condition associated with elevated LDL-C levels.

In a ninth aspect, present invention concerns a method for testing the efficacy of a compound in modulating cholesterol levels in a subject, comprising the steps:
(a) providing a rodent;
(b) administering an antibody or an antigen-binding fragment thereof which specifically binds PCSK9 to the rodent;
(c) administering a test compound to said rodent;
(d) determining in the rodent one or more of the parameters selected from the group consisting of: total cholesterol levels, LDL-C levels or HDL-C levels
   (i) before administration of the test compound to the rodent and
   (ii) after administration of the test compound to the rodent:,
(e) comparing the parameters obtained in (d)(ii) and (d)(ii)
   wherein a difference in the parameters obtained in (d) (ii) with the parameter obtained in (d) (ii) indicates that the test compound compound is efficacious in modulating cholesterol levels in a subject.

In a tenth aspect, present invention concerns an in vitro method for testing the efficacy of a compound in modulating cholesterol levels in a subject, comprising the steps:
(a) determining one or more of the parameters selected from the group consisting of: total cholesterol levels, LDL-C levels or HDL-C levels,
   (i) in a sample of a rodent obtained before administration of the test compound to the rodent, and
   (ii) in a sample of the same rodent obtained administration of the test compound, and
(b) comparing the parameters determined in (d)(ii) and (d)(ii)
   wherein the rodent has been administered an antibody or an antigen-binding fragment thereof which specifically binds PCSK9 in conjunction with the test administration compound and
   wherein a difference in the parameters obtained in (d) (ii) with the parameter obtained in (d) (ii) indicates that the test compound compound is efficacious in modulating cholesterol levels in a subject.

According to a preferred embodiment of the ninth or tenth aspect, a decreased level of cholesterol (total or LDL-C) and/or an increased level increase of the HDL-C level in (ii) in comparison to (i) indicates that the test compound is efficacious in the treatment or prevention of one or more of the diseases or disorders associated with elevated LDL-C levels in a subject.

According to another preferred embodiment of the ninth or tenth aspect an increased level of cholesterol (total or LDL-C) in (ii) in comparison to (i) indicates that the test compound has adverse effects and may promote, contribute to or trigger of one or more of the diseases or disorders associated with elevated LDL-C levels in a subject.

According to another preferred embodiment of the ninth and tenth aspect and other aspects of present invention making use of a test- and a control-rodent, the control rodent is from the same species and preferably also from the same strain as the test rodent. Moreover, it may be advantageous if the control- and test-rodents have the same gender and/or approximate age and/or health status and/or body weight. In an example both, test- and control-rodent are male syrian hamster.

According to one embodiment of the ninth and tenth aspect the test compound is a PCSK9-inhibitor, such as a PCSK9 antibody or an HMG-CoA reductase inhibitor, e.g. a statin.

According to a preferred embodiment of the different aspects of present invention in which an antibody is administered to a rodent (e.g. to a hamster), the antibody is administered to the rodent in an amount of about 0.5 mg/kg body weight to about 15 mg/kg body weight and preferably in a concentration of about 0.8 mg/kg body weight to about 13 mg/kg body weight and preferably in a concentration of about, 0.5 mg/kg, about 1 mg/kg body weight, about 2 mg/kg, about 3 mg/kg, about 4 mg/kg, about 5 mg/kg, about 6 mg/kg, about 7 mg/kg about 8 mg/kg about 9 mg/kg, 10 about mg/kg, about 11 mg/kg, about 12 mg/kg or about 13 mg/kg body weight. According to a further preferred embodiment, the antibody is administered to the rodent, preferably a hamster, in an amount of about 1 mg/kg body weight, about 3 mg/kg body weight or about 10 mg/kg body weight.

According to a preferred embodiment of the first, third, fifth, seventh or ninth aspect, the parameter or level of cholesterol is determined in a taken sample.

The determination of the cholesterol level such as the total cholesterol level, the HDL-cholesterol level or the LDL-cholesterol level is known in the art and comprises, for example colorimetric, photometric, fluorometric gravimetric or spectroscopic methods.

The taken sample of the rodent can be derived from any part (organ, (solid or liquid) tissue etc) of the rodent that typically contains cholesterol such as liver or body liquid, e.g. blood, plasma or serum. The skilled person knows how to obtain such samples (e.g. taking of blood) from rodents, e.g. from hamster, prepare them (if necessary) and determine the cholesterol level therein (e.g. using a commercial kit, see e.g. the example section).

Although present methods are conducted using a rodent, the outcome of each of the methods can be interpreted to be indicative for other animals or species than the animal or species used in the method, such as other rodents than the used species, other mammals than the used species and preferably indicative for humans.

The disease or condition associated with elevated LDL-C levels according to the aspects of present invention can be any disease or condition typically associated with elevated LDL-C levels and is preferably a condition associated with elevated LDL-C levels in humans, such as hypercholesterolemia, hyperlipidemia, dyslipidemia, atherosclerosis, cardiovascular diseases, particularly primary hypercholesterolemia, familial hypercholesterolemia. or hypercholesteremia which is uncontrolled by statins.

In an eleventh aspect, present invention is directed to a rodent for use in identifying a drug for the treatment of a disease associated with elevated cholesterol levels and preferably associated with elevated LDL-C levels, wherein the rodent has decreased PCSK9 levels in comparison to a control rodent.

In the different aspects and embodiments of the invention, a rodent has has decreased PCSK9 levels or decreased PCSK9 activity in comparison to a reference (preferably at least at 5%, 25%, 30%, 50%, 70% or at least 80% lower than the reference), e.g. in comparison to a reference rodent or reference value derived from average rodents, e.g. one or more reference rodents with average PCSK9 levels as representative for rodents of the same species and/or the same health condition and/or the same strain and/or the same gender. E.g. if the rodent is a hamster, such as a syrian hamster, such as a male syrian hamster, a rodent with decreased PCSK9 levels or activity has a PCSK9 level (preferably protein level) or activity (e.g. protein activity) that is significantly lower (e.g. at least 5%, 25%, 30%, 50%, 70% or at least 80% lower) than that at of average hamsters, preferably of average syrian hamsters, or of average male hamsters or of average male syrian hamsters or of average hamsters of the same health status (e.g. normolipidemic or healthy) or of average hamsters of about the same age and preferably having a significantly lower PCSK9 level or activity than average male syrian hamsters.

In a twelfth aspect, present invention is directed to the use of a rodent with decreased PCSK9 levels in comparison to a control rodent as a model system for determining the cholesterol-modulating effect and preferably of the cholesterol-lowering effect of a drug. In a preferred embodiment of the eleventh or twelfth aspect, the drug is an HMG-CoA reductase inhibitor such as a statin.

According to a preferred embodiment of the eleventh aspect, the rodent is for use in a method according to one of the aspects 1, 2, 7, 8, 9 or 10.

According to another preferred embodiment, the lowered PCSK-9 level or activity in the rodent with lowered PCSK9-levels or activity as used in different aspects of present invention, is caused by a genomic knock-out of PCSK9, a stable or transient knock-down of PCSK9 or administration of a PCSK-9 antagonist.

Antagonists of PCSK-9 comprise small molecules or biological molecules such as antibodies, non-antibody protein scaffolds (e.g. darpins, anticalins, nanobodies, affibodies etc.) or antagonistic nucleic acids such as double-stranded or single stranded DNA or RNA which, for example, inhibit the expression of the PCSK-9 gene or the activity of PCSK-9 and includes, without limitation, antisense nucleic acids, aptamers, siRNAs (small interfering RNAs) and ribozymes.

The nucleic acids, e.g. the antisense nucleic acids, can be synthesized chemically, e.g. in accordance with the phosphotriester method (see, for example, Uhlmann, E. & Peyman, A. (1990) Chemical Reviews, 90, 543-584). Aptamers are nucleic acids which bind with high affinity to a polypeptide, here PAK. Aptamers can be isolated by selection methods such as SELEX (see e.g. Jayasena (1999) Clin. Chem., 45, 1628-50; Klug and Famulok (1994) M. Mol. Biol. Rep., 20, 97-107; U.S. Pat. No. 5,582,981) from a large pool of different single-stranded RNA molecules. Aptamers can also be synthesized and selected in their mirror-image form, for example as the L-ribonucleotide (Nolte et al. (1996) Nat. Biotechnol., 14, 1116-9; Klussmann et al. (1996) Nat. Biotechnol., 14, 1112-5). Forms which have been isolated in this way enjoy the advantage that they are not degraded by naturally occurring ribonucleases and, therefore, possess greater stability.

A knock-out animal an animal, in which one or more genes are downregulated or turned off by means of a targeted mutation in the genome or a nucleic acid (e.g. a DNA vector, an oligonucleotide, or a siRNA) introduced transiently or stably into the animal. The generation of knock-out animals, especially of knock-out rodents is known in the art; the generation of PCSK-9 knock-out rodents thus lies within the skill of the artisan. An example of a knock-out animal can e.g. be taken from Mbikay M., et al., FEBS Letters, 2010, February 19, 584 (4): 701-6 that describes a PCSK9 knock-out mouse.

The downregulation of PCSK9 can be achieved by any type of modification of the animal or cell (stable or transient, preferably stable), that leads to a decrease of PCSK9 activity (i.e. its ability to PCSK9), PCSK9 transcript steady state level (i.e. by activation of PCSK9 transcription or transcript stabilisation) or PCSK9 protein steady state level (i.e. by activation of PCSK9 translation or its posttranslational processing; by modulation of PCSK9 posttranslational modification or by activation of its stabilisation or by inhibition of its degradation). This can for example be achieved by using dominant negative mutants of PCSK9, antisense oligonucleotides, RNAi constructs of PCSK9, by generating functional or genomic PCSK9 knock outs (which can e.g. be inducible) or other suitable techniques known within the state of the art. For an overview of the above techniques, see for example: Current protocols in Molecular biology (2000) J. G. Seidman, Chapter 23, Supplemtent 52, John Wiley and Sons, Inc.; Gene Targeting: a practical approach (1995), Editor: A. L. Joyner, IRL Press; Genetic Manipulation of Receptor Expression and Function, 2000; Antisense Therapeutics, 1996; Scherr et al, 2003.

The term “knock-down” as used herein also refers to the downregulation of PCSK-9 protein levels or activity in the rodent using siRNA.

In a thirteenth aspect, present invention concerns a method for the preparation of a rodent suitable for use as model system for determining the cholesterol-modulating effect and preferably of the cholesterol-lowering effect of a drug, the method comprising providing a rodent or a blastocyst of a rodent and lowering its PCSK9 level by means of a genomic knock-out of PCSK9, a stable or transient knock-down of PCSK9 or administration of a PCSK-9 antagonist.

In a preferred embodiment of the twelfth or thirteenth aspect, e lowered PCSK-9 activity or expression level in the rodent is caused by administration of a PCSK9 antagonist, preferably a specific PCSK-9 antibody to the rodent.

In a further preferred embodiment of the different aspects and embodiments of present invention, the rodent is selected from hamster, mouse, rat, guinea pig and rabbit and is preferably a hamster, more preferably a syrian hamster. According to a particularly preferred embodiment, the hamster is a male syrian hamster.

According to another preferred embodiment, the rodent is normolipidemic or hyperlipidemic and preferably normolipidemic.

In a fourteenth aspect, present invention concerns a rodent, preferably a hamster, obtained by a method according to the thirteenth aspect, and preferably obtained by administration of a PCSK-9 specific antibody to the rodent.

In a fifteenth aspect, present invention concerns a kit for conducting a method according to one of the aspects 1 to 10 comprising a rodent, preferably a hamster and a PCSK 9-specific antagonist, such as a PCSK9-specific antibody and optionally comprising one or more of the further components according to one of the aspects sixteen and seventeen.

In a sixteenth aspect, present invention concerns an article of manufacture comprising
(a) a packaging material or container;
(b) an antibody or an antigen-binding fragment thereof which specifically binds hPCSK9; and
(c) a data carrier such as a label or packaging insert contained within the packaging material containing instructions for carrying out a method according to one of aspects 1 to 10 for profiling or identifying compounds for use in the treatment or prevention of hypercholesterolemia, hyperlipidemia, dyslipidemia, atherosclerosis and cardiovascular diseases and optionally
(d) one or more buffers and/or reagents for determining total cholesterol levels, LDL-C levels or HDL-C levels in a sample.

In a seventeenth aspect, present invention concerns article of manufacture comprising
(a) a packaging material or container;
(b) reagents and buffers for determining total cholesterol levels, LDL-C levels or HDL-C levels in a sample; and
(c) a data carrier such as a label containing instructions for carrying out a method according to one of the aspects 1 to 10 and optionally (d) an antibody or an antigen-binding fragment thereof which specifically binds hPCSK9.

According to a preferred embodiment of the sixteenth or seventeenth aspect, the article of manufacture according comprises one or more rodents, such as a hamster, preferably a syrian hamster.

According to another preferred embodiment of the sixteenth or seventeenth aspect, the article of manufacture comprises

a data carrier, wherein the data carrier comprises information such as
(i) instructions for use of the antibody or fragment thereof
(ii) quality information such as information about the lot/batch number of the antibody or of the article of, the manufacturing or assembly site or the expiry or sell-by date, information concerning the correct storage or handling of the article,
(iii) information concerning the composition of the buffer(s), diluent(s), reagent(s) for determining the cholesterol levels or for use of the antibody,
(iv) information concerning the interpretation of information obtained when performing the above-mentioned methods,
(v) a warning concerning possible misinterpretations or wrong results when applying unsuitable methods, and/or
(vi) a warning concerning possible misinterpretations or wrong results when using unsuitable reagent(s) and/or buffer(s).

The rodent to be used for the different aspects of present invention is preferably selected from hamster, mouse, rat, guinea pig and rabbit and is more preferably a hamster. According to a particularly preferred embodiment, the rodent is a syrian hamster, even more preferably a male syrian hamster. According to another preferred embodiment of the different aspects of present invention, the rodent is a normolipidemic or hyperlipidemic and preferably normolipidemic rodent such as a normolipidemic hamster.

Several aspects and embodiments of the invention can be combined with each other. The skilled artisan will recognize other preferred embodiments resulting of suitable combinations of different aspects and embodiments of present invention.

### PREFERRED ANTIBODIES FOR PRACTICING THE PRESENT INVENTION

The following section describes functional and structural features of antibodies and antigen-binding fragments thereof that can be used for practicing all aspects of the present invention. Thus, expressions such as “in preferred embodiments”, “in some embodiments”, “in another preferred embodiment” and similar expressions should be understood as referring to embodiments of the first aspect of the present invention, the second aspect of the present invention, the third aspect of the present invention, the fourth aspect of the present invention, the fifth aspect of the present invention, the sixth aspect of the present invention, the seventh aspect of the present invention, the eighth aspect of the present invention, the ninth aspect of the present invention, the tenth aspect of the present invention, the eleventh aspect of the present invention, the twelfth aspect of the present invention, the thirteenth aspect of the present invention, the fourteenth aspect of the present invention, the fifteenth aspect of the present invention, the sixteenth aspect of the present invention and the seventeenth aspect of the present invention.

All antibodies or antigen-binding fragments thereof suitable for practicing the present invention specifically bind hPCSK9. In preferred embodiments of any aspect of the present invention, the antibody or antigen-binding fragment thereof is a recombinant human antibody or fragment thereof. In more specific embodiments, the antibody or antigen-binding fragment thereof is a fully human monoclonal antibody or antigen-binding fragment thereof that specifically binds hPCSK9 and neutralizes PCSK9 activity.

The mAbs usable in the present invention can be full-length (e.g., an IgG1 or IgG4 antibody) or may comprise only an antigen-binding portion (e.g., a Fab, F(ab′)₂ or scFv fragment), and may be modified to affect functionality, e.g., to eliminate residual effector functions (Reddy et al. (2000) J. Immunol. 164:1925-1933).

In one embodiment, the antibody or the antigen-binding fragment thereof is characterized as binding an epitope comprising amino acid residue 238 of hPCSK9 (SEQ ID NO:2). In a more specific embodiment, the antibody or antigen-binding fragment binds an epitope comprising one or more of amino acid residues at positions 238, 153, 159 and 343 of hPCSK9 (SEQ ID NO:2). In a more specific embodiment, the antibody or fragment thereof is characterized as binding an epitope which does not comprise an amino acid residue at positions 192, 194, 197 and/or 237 of SEQ ID NO: 2.

In one embodiment, the antibody or the antigen-binding fragment thereof is characterized as binding an epitope comprising amino acid residue 366 of hPCSK9 (SEQ ID NO: 2). In a more specific embodiment, the antibody or antigen-binding fragment binds an epitope comprising one or more of amino acid residues at positions 147, 366 and 380 of hPCSK9 (SEQ ID NO: 2). In a more specific embodiment, the antibody or antigen-binding fragment of an antibody is characterized as binding an epitope which does not comprise an amino acid residue at position 215 or 238 of SEQ ID NO: 2.

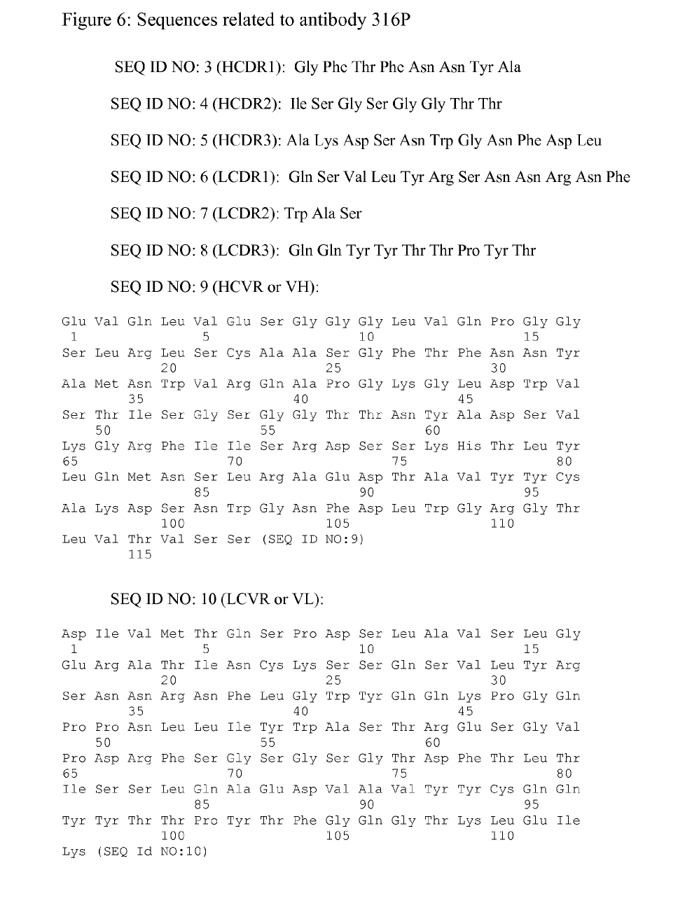

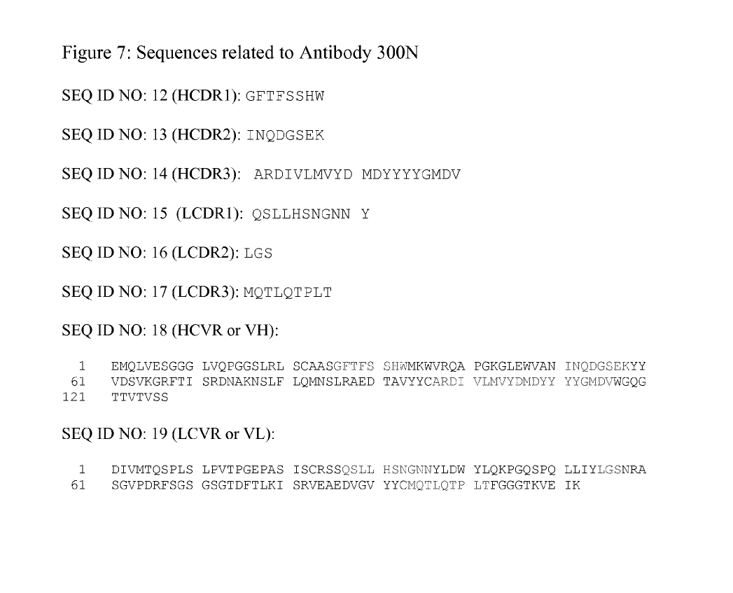

In one embodiment, the antibody or the antigen-binding fragment thereof comprises one or more of the sequences of antibody 316P or 300N as listed herein (see e.g. FIGS. 6 and 7).

In one embodiment, the antibody or the antigen-binding fragment thereof comprises the heavy chain variable region (HCVR), of SEQ ID NO: 9 or a substantially similar sequence thereof having at least 90%, at least 95%, at least 98% or at least 99% sequence identity.

In one embodiment, the antibody or the antigen-binding fragment thereof further comprises the light chain variable region (LCVR) of SEQ Id NO: 10 or a substantially similar sequence thereof having at least 90%, at least 95%, at least 98% or at least 99% sequence identity.

In specific embodiments, the antibody or the antigen-binding fragment thereof comprises HCVR amino acid sequence as shown in SEQ ID NO: 9 and an LCVR amino acid sequence as shown in SEQ ID NO: 10.

In preferred embodiments, the antibody or the antigen-binding fragment thereof comprises a heavy chain CDR3 (HCDR3) domain of SEQ ID NO: 5 or a substantially similar sequence thereof having at least 90%, at least 95%, at least 98% or at least 99% sequence identity; and/or a light chain CDR3 (LCDR3) domain of SEQ ID NO: 8, or substantially similar sequences thereof having at least 90%, at least 95%, at least 98% or at least 99% sequence identity. In one embodiment, the HCDR3/LCDR3 sequence pair is SEQ ID NO:5/8. In more preferred embodiments, the antibody or the antigen-binding fragment thereof comprises a HCDR3 domain as shown in SEQ ID NO: 5 and a LCDR3 domain as shown in SEQ ID NO: 8.

In a further embodiment, the antibody or the antigen-binding fragment thereof further comprises the heavy chain CDR1 (HCDR1) domain of SEQ ID NO: 3, or a substantially similar sequence thereof having at least 90%, at least 95%, at least 98% or at least 99% sequence identity; and/or the heavy chain CDR2 (HCDR2) domain of SEQ ID NO: 4 or a substantially similar sequence thereof having at least 90%, at least 95%, at least 98% or at least 99% sequence identity; and/or a light chain CDR1 (LCDR1) domain of SEQ ID NO: 6 or a substantially similar sequence thereof having at least 90%, at least 95%, at least 98% or at least 99% sequence identity; and/or a light chain CDR2 (LCDR2) domain of SEQ ID NO: 7, or a substantially similar sequence thereof having at least 90%, at least 95%, at least 98% or at least 99% sequence identity. In one embodiment, the heavy and light chain CDR sequences comprise a sequence selected from the group consisting of SEQ ID NO: 3, 4, 5, 6, 7, 8. In preferred embodiments, the antibody or antigen-binding fragment thereof comprises heavy and light chain CDR amino acid sequences as shown in SEQ ID NOs: 3, 4, 5, 6, 7 and 8.

In a related preferred embodiment, the antibody or antigen-binding fragment thereof comprises heavy and light chain CDR domains contained within the heavy and light chain sequence pair of SEQ ID NO:, 9/10.

In one embodiment, the antibody or the antigen-binding fragment thereof comprises the heavy chain variable region (HCVR or VH), of SEQ ID NO: 18 or a substantially similar sequence thereof having at least 90%, at least 95%, at least 98% or at least 99% sequence identity.

In one embodiment, the antibody or the antigen-binding fragment thereof further comprises the light chain variable region (LCVR or VL) of SEQ Id NO: 19 or a substantially similar sequence thereof having at least 90%, at least 95%, at least 98% or at least 99% sequence identity.

In specific embodiments, the antibody or the antigen-binding fragment thereof comprises HCVR amino acid sequence as shown in SEQ ID NO: 18 and an LCVR amino acid sequence as shown in SEQ ID NO: 19.

In preferred embodiments, the antibody or the antigen-binding fragment thereof comprises a heavy chain CDR3 (HCDR3) domain of SEQ ID NO: 14 or a substantially similar sequence thereof having at least 90%, at least 95%, at least 98% or at least 99% sequence identity; and/or a light chain CDR3 (LCDR3) domain of SEQ ID NO: 17, or substantially similar sequences thereof having at least 90%, at least 95%, at least 98% or at least 99% sequence identity. In one embodiment, the HCDR3/LCDR3 sequence pair is SEQ ID NO:14/17. In more preferred embodiments, the antibody or the antigen-binding fragment thereof comprises a HCDR3 domain as shown in SEQ ID NO: 14 and a LCDR3 domain as shown in SEQ ID NO: 17.

In a further embodiment, the antibody or the antigen-binding fragment thereof further comprises the heavy chain CDR1 (HCDR1) domain of SEQ ID NO: 12, or a substantially similar sequence thereof having at least 90%, at least 95%, at least 98% or at least 99% sequence identity; and/or the heavy chain CDR2 (HCDR2) domain of SEQ ID NO: 13 or a substantially similar sequence thereof having at least 90%, at least 95%, at least 98% or at least 99% sequence identity; and/or a light chain CDR1 (LCDR1) domain of SEQ ID NO: 15 or a substantially similar sequence thereof having at least 90%, at least 95%, at least 98% or at least 99% sequence identity; and/or a light chain CDR2 (LCDR2) domain of SEQ ID NO: 16, or a substantially similar sequence thereof having at least 90%, at least 95%, at least 98% or at least 99% sequence identity. In one embodiment, the heavy and light chain CDR sequences comprise a sequence selected from the group consisting of SEQ ID NO: 12, 13, 14, 15, 16 and 17. In preferred embodiments, the antibody or antigen-binding fragment thereof comprises heavy and light chain CDR amino acid sequences as shown in SEQ ID NOs: 12, 13, 14, 15, 16 and 17.

In a related preferred embodiment, the antibody or antigen-binding fragment thereof comprises heavy and light chain CDR domains contained within the heavy and light chain sequence pair of SEQ ID NO: 18/19.

In a further embodiment, the antibody or antigen-binding fragment thereof is a human anti-PCSK9 antibody or antigen-binding fragment thereof comprising a heavy chain variable region (HCVR) encoded by nucleotide sequence segments derived from V_{H}, D_{H} and J_{H} germline sequences, and a light chain variable region (LCVR) encoded by nucleotide sequence segments derived from V_{K} and J_{K} germline sequences, wherein the germline sequences are (a) V_{H} gene segment 3-23, D_{H} gene segment 7-27, J_{H} gene segment 2, V_{K} gene segment 4-1 and J_{K} gene segment 2; or (b) V_{H} gene segment 3-7, D_{H} gene segment 2-8, J_{H} gene segment 6, V_{K} gene segment 2-28 and J_{K} gene segment 4.

In preferred embodiments, the antibody or antigen-binding fragment thereof binds to the same epitope on hPCSK9 as an antibody comprising heavy and light chain CDR amino acid sequences as shown in SEQ ID NOs: 3, 4, 5, 6, 7, and 8.

In preferred embodiments, the antibody or antigen-binding fragment thereof competes for binding to hPCSK9 with an antibody comprising heavy and light chain CDR amino acid sequences as shown in SEQ ID NOs: 3, 4, 5, 6, 7, and 8.

The invention encompasses anti-PCSK9 antibodies having a modified glycosylation pattern. In some applications, modification to remove undesirable glycosylation sites may be useful, or e.g., removal of a fucose moiety to increase antibody dependent cellular cytotoxicity (ADCC) function (see Shield et al. (2002) JBC 277:26733). In other applications, modification of galactosylation can be made in order to modify complement dependent cytotoxicity (CDC).
Some preferred sequences related to the antibodies for practicing present invention:

```
SEQ ID NO: 3:
Gly Phe Thr Phe Asn Asn Tyr Ala
SEQ ID NO: 4:
Ile Ser Gly Ser Gly Gly Thr Thr
SEQ ID NO: 5:
Ala Lys Asp Ser Asn Trp Gly Asn Phe Asp Leu
SEQ ID NO: 6:
Gln Ser Val Leu Tyr Arg Ser Asn Asn Arg Asn Phe
SEQ ID NO: 7:
Trp Ala Ser
SEQ ID NO: 8:
Gln Gln Tyr Tyr Thr Thr Pro Tyr Thr
SEQ ID NO: 9:
Glu Val Gln Leu Val Glu Ser Gly Gly Gly Leu Val Gln Pro Gly Gly
1 5 10 15
Ser Leu Arg Leu Ser Cys Ala Ala Ser Gly Phe Thr Phe Asn Asn Tyr
                      20 25 30
Ala Met Asn Trp Val Arg Gln Ala Pro Gly Lys Gly Leu Asp Trp Val
        35 40 45
Ser Thr Ile Ser Gly Ser Gly Gly Thr Thr Asn Tyr Ala Asp Ser Val
    50 55 60
Lys Gly Arg Phe Ile Ile Ser Arg Asp Ser Ser Lys His Thr Leu Tyr
65 70 75 80
Leu Gln Met Asn Ser Leu Arg Ala Glu Asp Thr Ala Val Tyr Tyr Cys
                85 90 95
Ala Lys Asp Ser Asn Trp Gly Asn Phe Asp Leu Trp Gly Arg Gly Thr
                100 105 110
Leu Val Thr Val Ser Ser
        115
SEQ ID NO: 10:
Asp Ile Val Met Thr Gln Ser Pro Asp Ser Leu Ala Val Ser Leu Gly
1 5 10 15
Glu Arg Ala Thr Ile Asn Cys Lys Ser Ser Gln Ser Val Leu Tyr Arg
              20 25 30
Ser Asn Asn Arg Asn Phe Leu Gly Trp Tyr Gln Gln Lys Pro Gly Gln
          35 40 45
Pro Pro Asn Leu Leu Ile Tyr Trp Ala Ser Thr Arg Glu Ser Gly Val
    50 55 60
Pro Asp Arg Phe Ser Gly Ser Gly Ser Gly Thr Asp Phe Thr Leu Thr
65 70 75 80
Ile Ser Ser Leu Gln Ala Glu Asp Val Ala Val Tyr Tyr Cys Gln Gln
                    85 90 95
Tyr Tyr Thr Thr Pro Tyr Thr Phe Gly Gln Gly Thr Lys Leu Glu Ile
              100 105 110
Lys
```

### Preparation of Human Antibodies

Methods for generating human antibodies in transgenic mice are known (see for example, U.S. Pat. No. 6,596,541, Regeneron Pharmaceuticals, VELOCIMMUNE™). The VELOCIMMUNE™ technology involves generation of a transgenic mouse having a genome comprising human heavy and light chain variable regions operably linked to endogenous mouse constant region loci such that the mouse produces an antibody comprising a human variable region and a mouse constant region in response to antigenic stimulation. The DNA encoding the variable regions of the heavy and light chains of the antibody are isolated and operably linked to DNA encoding the human heavy and light chain constant regions. The DNA is then expressed in a cell capable of expressing the fully human antibody. In specific embodiment, the cell is a CHO cell.

Antibodies may be therapeutically useful in blocking a ligand-receptor interaction or inhibiting receptor component interaction, rather than by killing cells through fixation of complement and participation in complement-dependent cytotoxicity (CDC), or killing cells through antibody-dependent cell-mediated cytotoxicity (ADCC). The constant region of an antibody is thus important in the ability of an antibody to fix complement and mediate cell-dependent cytotoxicity. Thus, the isotype of an antibody may be selected on the basis of whether it is desirable for the antibody to mediate cytotoxicity.

Human antibodies can exist in two forms that are associated with hinge heterogeneity. In one form, an antibody molecule comprises a stable four-chain construct of approximately 150-160 kDa in which the dimers are held together by an interchain heavy chain disulfide bond. In a second form, the dimers are not linked via inter-chain disulfide bonds and a molecule of about 75-80 kDa is formed composed of a covalently coupled light and heavy chain (half-antibody). These forms have been extremely difficult to separate, even after affinity purification.

The frequency of appearance of the second form in various intact IgG isotypes is due to, but not limited to, structural differences associated with the hinge region isotype of the antibody. A single amino acid substitution in the hinge region of the human IgG4 hinge can significantly reduce the appearance of the second form (Angal et al. (1993) Molecular Immunology 30:105) to levels typically observed using a human IgG1 hinge. The instant invention encompasses antibodies having one or more mutations in the hinge, CH2 or CH3 region which may be desirable, for example, in production, to improve the yield of the desired antibody form.

Generally, a VELOCIMMUNE™ mouse is challenged with the antigen of interest, and lymphatic cells (such as B-cells) are recovered from the mice that express antibodies. The lymphatic cells may be fused with a myeloma cell line to prepare immortal hybridoma cell lines, and such hybridoma cell lines are screened and selected to identify hybridoma cell lines that produce antibodies specific to the antigen of interest. DNA encoding the variable regions of the heavy chain and light chain may be isolated and linked to desirable isotypic constant regions of the heavy chain and light chain. Such an antibody protein may be produced in a cell, such as a CHO cell. Alternatively, DNA encoding the antigen-specific chimeric antibodies or the variable domains of the light and heavy chains may be isolated directly from antigen-specific lymphocytes.

Initially, high affinity chimeric antibodies are isolated having a human variable region and a mouse constant region. As described below, the antibodies are characterized and selected for desirable characteristics, including affinity, selectivity, epitope, etc. The mouse constant regions are replaced with a desired human constant region to generate the fully human antibody of the invention, for example wild-type or modified IgG1 or IgG4. While the constant region selected may vary according to specific use, high affinity antigen-binding and target specificity characteristics reside in the variable region.

### Epitope Mapping and Related Technologies

To screen for antibodies that bind to a particular epitope (e.g., those which block binding of IgE to its high affinity receptor), a routine cross-blocking assay such as that described Antibodies, Harlow and Lane (Cold Spring Harbor Press, Cold Spring Harb., NY) can be performed. Other methods include alanine scanning mutants, peptide blots (Reineke (2004) Methods Mol Biol 248:443-63) (herein specifically incorporated by reference in its entirety), or peptide cleavage analysis. In addition, methods such as epitope excision, epitope extraction and chemical modification of antigens can be employed (Tomer (2000) Protein Science 9: 487-496) (herein specifically incorporated by reference in its entirety).

The term “epitope” refers to a site on an antigen to which B and/or T cells respond. B-cell epitopes can be formed both from contiguous amino acids or noncontiguous amino acids juxtaposed by tertiary folding of a protein. Epitopes formed from contiguous amino acids are typically retained on exposure to denaturing solvents, whereas epitopes formed by tertiary folding are typically lost on treatment with denaturing solvents. An epitope typically includes at least 3, and more usually, at least 5 or 8-10 amino acids in a unique spatial conformation.

Modification-Assisted Profiling (MAP), also known as Antigen Structure-based Antibody Profiling (ASAP) is a method that categorizes large numbers of monoclonal antibodies (mAbs) directed against the same antigen according to the similarities of the binding profile of each antibody to chemically or enzymatically modified antigen surfaces (US 2004/0101920, herein specifically incorporated by reference in its entirety). Each category may reflect a unique epitope either distinctly different from or partially overlapping with epitope represented by another category. This technology allows rapid filtering of genetically identical mAbs, such that characterization can be focused on genetically distinct mAbs. When applied to hybridoma screening, MAP may facilitate identification of rare hybridoma clones that produce mAbs having the desired characteristics. MAP may be used to sort the anti-PCSK9 mAbs of the invention into groups of mAbs binding different epitopes.

In various embodiments, the anti-hPCSK9 antibody or antigen-binding fragment of an antibody binds an epitope within the catalytic domain, which is about 153 to 425 of SEQ ID NO:2); more specifically, an epitope from about 153 to about 250 or from about 250 to about 425; more specifically, the antibody or antibody fragment of the invention binds an epitope within the fragment from about 153 to about 208, from about 200 to about 260, from about 250 to about 300, from about 275 to about 325, from about 300 to about 360, from about 350 to about 400, and/or from about 375 to about 425.

In various embodiments, the anti-hPCSK9 antibody or antigen-binding fragment of an antibody binds an epitope within the propeptide domain (residues 31 to 152 of SEQ ID NO:2); more specifically, an epitope from about residue 31 to about residue 90 or from about residue 90 to about residue 152; more specifically, the antibody or antibody fragment of the invention binds an epitope within the fragment from about residue 31 to about residue 60, from about residue 60 to about residue 90, from about residue 85 to about residue 110, from about residue 100 to about residue 130, from about residue 125 to about residue 150, from about residue 135 to about residue 152, and/or from about residue 140 to about residue 152.

In some embodiments, the anti-hPCSK9 antibody or antigen-binding fragment of an antibody binds an epitope within the C-terminal domain, (residues 426 to 692 of SEQ ID NO:2); more specifically, an epitope from about residue 426 to about residue 570 or from about residue 570 to about residue 692; more specifically, the antibody or antibody fragment of the invention binds an epitope within the fragment from about residue 450 to about residue 500, from about residue 500 to about residue 550, from about residue 550 to about residue 600, and/or from about residue 600 to about residue 692.

In some embodiments, the antibody or antibody fragment binds an epitope which includes more than one of the enumerated epitopes within the catalytic, propeptide or C-terminal domain, and/or within two or three different domains (for example, epitopes within the catalytic and C-terminal domains, or within the propeptide and catalytic domains, or within the propeptide, catalytic and C-terminal domains.

In some embodiments, the antibody or antigen-binding fragment binds an epitope on hPCSK9 comprising amino acid residue 238 of hPCSK9 (SEQ ID NO:2). Experimental results (see US 2010/0166768) showed that when D238 was mutated, the K_{D} of mAb 316P exhibited >400-fold reduction in binding affinity (˜1×10⁻⁹ M to ˜410×10⁻⁹ M) and T_{1/2} decreased >30-fold (from ˜37 to ˜1 min). In a specific embodiment, the mutation was D238R. In specific embodiments, the antibody or antigen-binding fragment of the invention binds an epitope of hPCSK9 comprising two or more of amino acid residues at positions 153, 159, 238 and 343.

As shown before (see US 2010/0166768), a mutation in amino acid residue 153, 159 or 343 resulted in about a 5- to 10-fold decrease in affinity or similar shortening in T_{1/2}. In specific embodiments, the mutation was S153R, E159R and/or D343R.

In some embodiments, the antibody or antigen-binding fragment binds an epitope on hPCSK9 comprising amino acid residue 366 of hPCSK9 (SEQ ID NO:2). Experimental results (see US 2010/0166768) showed that when E366 was mutated, the affinity of mAb 300N exhibited about 50-fold decrease (˜0.7×10⁻⁹ M to ˜36×10⁻⁹ M) and a similar shortening in T_{1/2} (from ˜120 to ˜2 min). In a specific embodiment, the mutation is E366K.

The present invention includes anti-PCSK9 antibodies that bind to the same epitope as any of the specific exemplary antibodies described herein. Likewise, the present invention also includes anti-PCSK9 antibodies that compete for binding to PCSK9 or a PCSK9 fragment with any of the specific exemplary antibodies described herein.

One can easily determine whether an antibody binds to the same epitope as, or competes for binding with, a reference anti-PCSK9 antibody by using routine methods known in the art. For example, to determine if a test antibody binds to the same epitope as a reference anti-PCSK9 antibody of the invention, the reference antibody is allowed to bind to a PCSK9 protein or peptide under saturating conditions. Next, the ability of a test antibody to bind to the PCSK9 molecule is assessed. If the test antibody is able to bind to PCSK9 following saturation binding with the reference anti-PCSK9 antibody, it can be concluded that the test antibody binds to a different epitope than the reference anti-PCSK9 antibody. On the other hand, if the test antibody is not able to bind to the PCSK9 molecule following saturation binding with the reference anti-PCSK9 antibody, then the test antibody may bind to the same epitope as the epitope bound by the reference anti-PCSK9 antibody of the invention.

To determine if an antibody competes for binding with a reference anti-PCSK9 antibody, the above-described binding methodology is performed in two orientations: In a first orientation, the reference antibody is allowed to bind to a PCSK9 molecule under saturating conditions followed by assessment of binding of the test antibody to the PCSK9 molecule. In a second orientation, the test antibody is allowed to bind to a PCSK9 molecule under saturating conditions followed by assessment of binding of the reference antibody to the PCSK9 molecule. If, in both orientations, only the first (saturating) antibody is capable of binding to the PCSK9 molecule, then it is concluded that the test antibody and the reference antibody compete for binding to PCSK9. As will be appreciated by a person of ordinary skill in the art, an antibody that competes for binding with a reference antibody may not necessarily bind to the identical epitope as the reference antibody, but may sterically block binding of the reference antibody by binding an overlapping or adjacent epitope.

Two antibodies bind to the same or overlapping epitope if each competitively inhibits (blocks) binding of the other to the antigen. That is, a 1-, 5-, 10-, 20- or 100-fold excess of one antibody inhibits binding of the other by at least 50% but preferably 75%, 90% or even 99% as measured in a competitive binding assay (see, e.g., Junghans et al., Cancer Res. 1990 50:1495-1502). Alternatively, two antibodies have the same epitope if essentially all amino acid mutations in the antigen that reduce or eliminate binding of one antibody reduce or eliminate binding of the other. Two antibodies have overlapping epitopes if some amino acid mutations that reduce or eliminate binding of one antibody reduce or eliminate binding of the other.

Additional routine experimentation (e.g., peptide mutation and binding analyses) can then be carried out to confirm whether the observed lack of binding of the test antibody is in fact due to binding to the same epitope as the reference antibody or if steric blocking (or another phenomenon) is responsible for the lack of observed binding. Experiments of this sort can be performed using ELISA, RIA, surface plasmon resonance, flow cytometry or any other quantitative or qualitative antibody-binding assay available in the art.

In a specific embodiment, the invention comprises an anti-PCSK9 antibody or antigen binding fragment of an antibody that binds an PCSK9 protein of SEQ ID NO:2, wherein the binding between the antibody or fragment thereof to PCSK9 and a variant PCSK9 protein is less than 50% of the binding between the antibody or fragment and the PCSK9 protein of SEQ ID NO:2. In one specific embodiment, the variant PCSK9 protein comprises at least one mutation of a residue at a position selected from the group consisting of 153, 159, 238 and 343. In a more specific embodiment, the at least one mutation is S153R, E159R, D238R, and/or D343R. In another specific embodiment, the variant PCSK9 protein comprises at least one mutation of a residue at a position selected from the group consisting of 366. In one specific embodiment, the variant PCSK9 protein comprises at least one mutation of a residue at a position selected from the group consisting of 147, 366 and 380. In a more specific embodiment, the mutation is S147F, E366K and V380M.

### Immunoconjugates

The invention encompasses a human anti-PCSK9 monoclonal antibody conjugated to a therapeutic moiety (“immunoconjugate”), such as a cytotoxin, a chemotherapeutic drug, an immunosuppressant or a radioisotope. Cytotoxin agents include any agent that is detrimental to cells. Examples of suitable cytotoxin agents and chemotherapeutic agents for forming immunoconjugates are known in the art, see for example, WO 05/103081.

### Bispecifics

The antibodies of the present invention may be monospecific, bispecific, or multispecific. Multispecific mAbs may be specific for different epitopes of one target polypeptide or may contain antigen-binding domains specific for more than one target polypeptide. See, e.g., Tutt et al. (1991) J. Immunol. 147:60-69. The human anti-PCSK9 mAbs can be linked to or co-expressed with another functional molecule, e.g., another peptide or protein. For example, an antibody or fragment thereof can be functionally linked (e.g., by chemical coupling, genetic fusion, noncovalent association or otherwise) to one or more other molecular entities, such as another antibody or antibody fragment, to produce a bispecific or a multispecific antibody with a second binding specificity.

An exemplary bi-specific antibody format that can be used in the context of the present invention involves the use of a first immunoglobulin (Ig) CH3 domain and a second Ig CH3 domain, wherein the first and second Ig CH3 domains differ from one another by at least one amino acid, and wherein at least one amino acid difference reduces binding of the bispecific antibody to Protein A as compared to a bi-specific antibody lacking the amino acid difference.

In one embodiment, the first Ig CH3 domain binds Protein A and the second Ig CH3 domain contains a mutation that reduces or abolishes Protein A binding such as an H95R modification (by IMGT exon numbering; H435R by EU numbering). The second CH3 may further comprise a Y96F modification (by IMGT; Y436F by EU). Further modifications that may be found within the second CH3 include: D16E, L18M, N44S, K52N, V57M, and V82I (by IMGT; D356E, L358M, N384S, K392N, V397M, and V422I by EU) in the case of IgG1 antibodies; N44S, K52N, and V82I (IMGT; N384S, K392N, and V422I by EU) in the case of IgG2 antibodies; and Q15R, N44S, K52N, V57M, R69K, E79Q, and V82I (by IMGT; Q355R, N384S, K392N, V397M, R409K, E419Q, and V422I by EU) in the case of IgG4 antibodies.

Variations on the bi-specific antibody format described above are contemplated within the scope of the present invention.

### Bioequivalents

The anti-PCSK9 antibodies and antibody fragments of the present invention encompass proteins having amino acid sequences that vary from those of the described mAbs, but that retain the ability to bind human PCSK9. Such variant mAbs and antibody fragments comprise one or more additions, deletions, or substitutions of amino acids when compared to parent sequence, but exhibit biological activity that is essentially equivalent to that of the described mAbs. Likewise, the anti-PCSK9 antibody-encoding DNA sequences of the present invention encompass sequences that comprise one or more additions, deletions, or substitutions of nucleotides when compared to the disclosed sequence, but that encode an anti-PCSK9 antibody or antibody fragment that is essentially bioequivalent to an anti-PCSK9 antibody or antibody fragment of the invention. Examples of such variant amino acid and DNA sequences are discussed above.

Two antigen-binding proteins, or antibodies, are considered bioequivalent if, for example, they are pharmaceutical equivalents or pharmaceutical alternatives whose rate and extent of absorption do not show a significant difference when administered at the same molar dose under similar experimental conditions, either single does or multiple dose. Some antibodies will be considered equivalents or pharmaceutical alternatives if they are equivalent in the extent of their absorption but not in their rate of absorption and yet may be considered bioequivalent because such differences in the rate of absorption are intentional and are reflected in the labeling, are not essential to the attainment of effective body drug concentrations on, e.g., chronic use, and are considered medically insignificant for the particular drug product studied. In one embodiment, two antigen-binding proteins are bioequivalent if there are no clinically meaningful differences in their safety, purity, and potency.

In one embodiment, two antigen-binding proteins are bioequivalent if a patient can be switched one or more times between the reference product and the biological product without an expected increase in the risk of adverse effects, including a clinically significant change in immunogenicity, or diminished effectiveness, as compared to continued therapy without such switching.

In one embodiment, two antigen-binding proteins are bioequivalent if they both act by a common mechanism or mechanisms of action for the condition or conditions of use, to the extent that such mechanisms are known.

Bioequivalence may be demonstrated by in vivo and in vitro methods. Bioequivalence measures include, e.g., (a) an in vivo test in humans or other mammals, in which the concentration of the antibody or its metabolites is measured in blood, plasma, serum, or other biological fluid as a function of time; (b) an in vitro test that has been correlated with and is reasonably predictive of human in vivo bioavailability data; (c) an in vivo test in humans or other mammals in which the appropriate acute pharmacological effect of the antibody (or its target) is measured as a function of time; and (d) in a well-controlled clinical trial that establishes safety, efficacy, or bioavailability or bioequivalence of an antibody.

Bioequivalent variants of anti-PCSK9 antibodies of the invention may be constructed by, for example, making various substitutions of residues or sequences or deleting terminal or internal residues or sequences not needed for biological activity. For example, cysteine residues not essential for biological activity can be deleted or replaced with other amino acids to prevent formation of unnecessary or incorrect intramolecular disulfide bridges upon renaturation.

### Therapeutic Administration and Formulations

The invention provides therapeutic compositions comprising the anti-PCSK9 antibodies or antigen-binding fragments thereof of the present invention. The administration of therapeutic compositions in accordance with the invention will be administered with suitable carriers, excipients, and other agents that are incorporated into formulations to provide improved transfer, delivery, tolerance, and the like. A multitude of appropriate formulations can be found in the formulary known to all pharmaceutical chemists: Remington's Pharmaceutical Sciences, Mack Publishing Company, Easton, Pa. These formulations include, for example, powders, pastes, ointments, jellies, waxes, oils, lipids, lipid (cationic or anionic) containing vesicles (such as LIPOFECTIN™), DNA conjugates, anhydrous absorption pastes, oil-in-water and water-in-oil emulsions, emulsions carbowax (polyethylene glycols of various molecular weights), semi-solid gels, and semi-solid mixtures containing carbowax. See also Powell et al. “Compendium of excipients for parenteral formulations” PDA (1998) J Pharm Sci Technol 52:238-311.

The dose may vary depending upon the age and the size of a subject to be administered, target disease, conditions, route of administration, and the like. When the antibody of the present invention is used within the different aspects of present invention, in a rodent, it is advantageous to administer it intravenously or subcutaneously to the rodent and preferably subcutaneously.

It is suitable to administer the antibody to a rodent (e.g. to a hamster) in an amount of about 0.5 mg/kg body weight to about 15 mg/kg body weight and preferably in a concentration of about 0.8 mg/kg body weight to about 13 mg/kg body weight and preferably in a concentration of about, 0.5 mg/kg, about 1 mg/kg body weight, about 2 mg/kg, about 3 mg/kg, about 4 mg/kg, about 5 mg/kg, about 6 mg/kg, about 7 mg/kg about 8 mg/kg about 9 mg/kg, 10 about mg/kg, about 11 mg/kg, about 12 mg/kg or about 13 mg/kg body weight. According to a further preferred embodiment, the antibody is administered to the rodent, preferably a hamster, in an amount of about 1 mg/kg body weight, about 3 mg/kg body weight or about 10 mg/kg body weight.

Various delivery systems are known and can be used to administer the pharmaceutical composition of the invention, e.g., encapsulation in liposomes, microparticles, microcapsules, recombinant cells capable of expressing the mutant viruses, receptor mediated endocytosis (see, e.g., Wu et al. (1987) J. Biol. Chem. 262:4429-4432). Methods of introduction include, but are not limited to, intradermal, intramuscular, intraperitoneal, intravenous, subcutaneous, intranasal, epidural, and oral routes. The composition may be administered by any convenient route, for example by infusion or bolus injection, by absorption through epithelial or mucocutaneous linings (e.g., oral mucosa, rectal and intestinal mucosa, etc.) and may be administered together with other biologically active agents. Administration can be systemic or local.

The pharmaceutical composition can be also delivered in a vesicle, in particular a liposome (see Langer (1990) Science 249:1527-1533; Treat et al. (1989) in Liposomes in the Therapy of Infectious Disease and Cancer, Lopez Berestein and Fidler (eds.), Liss, New York, pp. 353-365; Lopez-Berestein, ibid., pp. 317-327; see generally ibid.). In certain situations, the pharmaceutical composition can be delivered in a controlled release system. In one embodiment, a pump may be used (see Langer, supra; Sefton (1987) CRC Crit. Ref. Biomed. Eng. 14:201). In another embodiment, polymeric materials can be used; see, Medical Applications of Controlled Release, Langer and Wise (eds.), CRC Pres., Boca Raton, Fla. (1974). In yet another embodiment, a controlled release system can be placed in proximity of the composition's target, thus requiring only a fraction of the systemic dose (see, e.g., Goodson, in Medical Applications of Controlled Release, supra, vol. 2, pp. 115-138, 1984).

The injectable preparations may include dosage forms for intravenous, subcutaneous, intracutaneous and intramuscular injections, drip infusions, etc. These injectable preparations may be prepared by methods publicly known. For example, the injectable preparations may be prepared, e.g., by dissolving, suspending or emulsifying the antibody or its salt described above in a sterile aqueous medium or an oily medium conventionally used for injections. As the aqueous medium for injections, there are, for example, physiological saline, an isotonic solution containing glucose and other auxiliary agents, etc., which may be used in combination with an appropriate solubilizing agent such as an alcohol (e.g., ethanol), a polyalcohol (e.g., propylene glycol, polyethylene glycol), a nonionic surfactant [e.g., polysorbate 80, HCO-50 (polyoxyethylene (50 mol) adduct of hydrogenated castor oil)], etc. As the oily medium, there are employed, e.g., sesame oil, soybean oil, etc., which may be used in combination with a solubilizing agent such as benzyl benzoate, benzyl alcohol, etc. The injection thus prepared is preferably filled in an appropriate ampoule. A pharmaceutical composition of the present invention can be delivered subcutaneously or intravenously with a standard needle and syringe. In addition, with respect to subcutaneous delivery, a pen delivery device readily has applications in delivering a pharmaceutical composition of the present invention. Such a pen delivery device can be reusable or disposable. A reusable pen delivery device generally utilizes a replaceable cartridge that contains a pharmaceutical composition. Once all of the pharmaceutical composition within the cartridge has been administered and the cartridge is empty, the empty cartridge can readily be discarded and replaced with a new cartridge that contains the pharmaceutical composition. The pen delivery device can then be reused. In a disposable pen delivery device, there is no replaceable cartridge. Rather, the disposable pen delivery device comes prefilled with the pharmaceutical composition held in a reservoir within the device. Once the reservoir is emptied of the pharmaceutical composition, the entire device is discarded.

Numerous reusable pen and autoinjector delivery devices have applications in the subcutaneous delivery of a pharmaceutical composition of the present invention. Examples include, but certainly are not limited to AUTOPEN™ (Owen Mumford, Inc., Woodstock, UK), DISETRONIC™ pen (Disetronic Medical Systems, Burghdorf, Switzerland), HUMALOG MIX 75/25™ pen, HUMALOG™ pen, HUMALIN 70/30™ pen (Eli Lilly and Co., Indianapolis, Ind.), NOVOPEN™ I, II and III (Novo Nordisk, Copenhagen, Denmark), NOVOPEN JUNIOR™ (Novo Nordisk, Copenhagen, Denmark), BD™ pen (Becton Dickinson, Franklin Lakes, N.J.), OPTIPEN™, OPTIPEN PRO™, OPTIPEN STARLET™, and OPTICLIK™ (sanofi-aventis, Frankfurt, Germany), to name only a few. Examples of disposable pen delivery devices having applications in subcutaneous delivery of a pharmaceutical composition of the present invention include, but certainly are not limited to the SOLOSTAR™ pen (sanofi-aventis), the FLEXPEN™ (Novo Nordisk), and the KWIKPEN™ (Eli Lilly).

Advantageously, the pharmaceutical compositions for oral or parenteral use described above are prepared into dosage forms in a unit dose suited to fit a dose of the active ingredients. Such dosage forms in a unit dose include, for example, tablets, pills, capsules, injections (ampoules), suppositories, etc. The amount of the aforesaid antibody contained is generally about 4 to about 500 mg or from about 5 to about 500 mg per dosage form in a unit dose; especially in the form of injection, it is preferred that the aforesaid antibody is contained in about 5 to about 100 mg or about 5 to 400 mg (such as from about 50 to about 200 mg per 1 ml injection solution) and in about 10 to about 250 mg or to about 500 mg for the other dosage forms.

The invention provides therapeutic methods in which the antibody or antibody fragment of the invention is useful to treat hypercholesterolemia associated with a variety of conditions involving hPCSK9. The anti-PCSK9 antibodies or antibody fragments of the invention are particularly useful for the treatment of hypercholesterolemia and the like. Combination therapies may include the anti-PCSK9 antibody of the invention with, for example, one or more of any agent that (1) induces a cellular depletion of cholesterol synthesis by inhibiting 3-hydroxy-3-methylglutaryl (HMG)-coenzyme A (CoA) reductase, such as cerivastatin, atorvastatin, simvastatin, pitavastatin, rosuvastatin, fluvastatin, lovastatin, pravastatin; (2) inhibits cholesterol uptake and or bile acid re-absorption; (3) increase lipoprotein catabolism (such as niacin); and activators of the LXR transcription factor that plays a role in cholesterol elimination such as 22-hydroxycholesterol or fixed combinations such as ezetimibe plus simvastatin; a statin with a bile resin (e.g., cholestyramine, colestipol, colesevelam), a fixed combination of niacin plus a statin (e.g., niacin with lovastatin); or with other lipid lowering agents such as omega-3-fatty acid ethyl esters (for example, omacor).

### EXAMPLES

The following examples are put forth so as to provide those of ordinary skill in the art with a complete disclosure and description of how to make and use the methods and compositions of the invention, and are not intended to limit the scope of what the inventors regard as their invention. Efforts have been made to ensure accuracy with respect to numbers used but some experimental errors and deviations should be accounted for. Unless indicated otherwise, molecular weight is average molecular weight, temperature is in degrees Centigrade, and pressure is at or near atmospheric.

### Active Compounds

### Antibody 316P

Antibody 316P is a fully human antibody comprising a HCVR as shown in SEQ ID NO: 9 and LCVR as shown in SEQ ID NO: 10 of the sequence listing. The CDR sequences are shown in SEQ ID NOs: 3, 4, and 5 (CDR1, CDR2, CDR3 of the heavy chain) as well as in SEQ ID NOs: 6, 7, and 8 (CDR1, CDR2, CDR3 of the light chain).

Antibody 316P is a human monoclonal anti-PCSK9 antibody derived from VelocImmune® technology that blocks the interaction between PCSK9 and LDLR. It antagonizes PCSK9-mediated down-regulation of LDL uptake via LDLR in HepG2 cells and has a high binding affinity for PCSK9, both in humans and in hamsters. The present investigation was divided into 2 studies: the first was designed to assess the dose response, safety and pharmacokinetics of a single subcutaneous (s.c.) injection of 316P in normolipidemic male Syrian hamsters (Mesocricetus auratus); the second study was designed to investigate the effects of a single s.c. injection of 316P and a twice-daily dose of atorvastatin, both alone and in combination with each other, on circulating levels of LDL-C and other lipids. The effects of each treatment on safety were also assessed.

### Methods and Statistics

### Animals and Treatments

Male Syrian hamsters (strain RjHan: AURA in Study 1 and Bio™ F₁B in Study 2), weighing between 90 and 100 g, were purchased from Janvier, Germany, and were allowed to acclimatize for a period of 7 days before entering the study. All animals were housed under controlled temperature (19-21° C.), humidity (55%) and 12 hours reversed dark/light cycle (light between 15:00 and 3:00), and had free access to water and standard hamster diet (Ssniff® Ha, Spezialitäten GmbH, Soest, Germany). The anti-hPCSK9 antibody 316P (Lot. 02-090211, stock 25.5 mg/mL) was expressed and purified at Regeneron Pharmaceuticals Inc. (Tarrytown, N.Y., USA) and was administered by s.c. injection on Day 0 using phosphate buffered saline (PBS) as a vehicle. Atorvastatin (Sortis 40 mg/tablet) was purchased from Pfizer Inc and was administered twice daily (morning and afternoon) orally (p.o.) for 7 days using a mixture of 0.5% hydroxyethyl cellulose, ethanol and solutol as a solvent. The animal studies were approved by the Sanofi-Aventis Deutschland GmbH institutional animal care and use committee. The institution is AAALAC accredited³⁸.

### Experimental Design

The investigation was divided into 2 studies. Eight-week-old animals were grouped according to weight prior to randomization and Study 1 animals (6 per group) received either s.c. PBS control, a single s.c. dose of 316P/PBS 1, 3, or 10 mg/kg on Day 0, or no treatment. Study 2 animals (10 per group) received either s.c. or p.o. PBS control, a single s.c. injection of 316P/PBS 10 mg/kg on Day 0, a twice-daily p.o. dose of atorvastatin 10 or 20 mg/kg with or without a single s.c. injection of 316P/PBS 10 mg/kg, or no treatment. For each study, body weight and food consumption (weight changes in chow) were measured once a week, starting 1 week before dosing. Blood was drawn from the retro-orbital vein plexus under Isofrluran CP® (CP Pharama, Burgdorf, Germany) oxygen/nitric oxide anaesthesia (3.5%, 2:1) between 09:00 and 11:00. In Study 1, samples were taken on Days −6, 1, 7, 14, 21, and 28; in Study 2, samples were taken on Days −7, 0, 3, and 7. Serum was obtained after centrifugation using Serum-Gel® tubes (Sarstedt, Nümbrecht, Germany). Serum levels of total cholesterol (TC), LDL-C, HDL-cholesterol (HDL-C), and TGs were measured on a Hitachi 912 analyzer, using the respective Roche clinical chemistry kits for human diagnostics.

At the end of each study, animals were sacrificed in a nonfasted state and liver was immediately weighed and shock frozen on dry ice. To measure liver cholesterol, TG and phospholipids, liver lipids were extracted using dichlormethan/methanol (v/v 2:1) and analyzed using a modification of a method described elsewhere.³⁹ Serum 316P levels were measured by sandwich ELISA using goat antihuman Fc antibody for capture and a horseradish peroxidase-conjugated goat anti-hFc antibody developed at Regeneron for detection. In Study 2, changes in hepatic LDLR levels were assessed by Western blot analysis. Concentrations of alanine amino transferase (ALT), aspartate amino transferase (AST), alkaline phosphatase (AP), and creatine kinase (CK) were determined on a Hitachi 912 analyzer, using the respective Roche clinical chemistry kits for human diagnostics.

### Statistical Analysis

All data are expressed as means±standard error of the mean (SEM) and were tested for normality and variance homogeneity before analysis. The effects of active treatment versus control were compared using 1-way analysis of variance (ANOVA), followed by the Kruskal-Wallis test or 2-way ANOVA followed by the Dunnett test. In Study 1, the vehicle-treated control versus treatment-naive control group was analyzed separately using 2-way ANOVA followed by the Dunnett test. A P value of ≦0.05 was considered statistically significant. Food consumption and safety parameters (AST, ALT, AP, and CK) were descriptively evaluated.

### Results

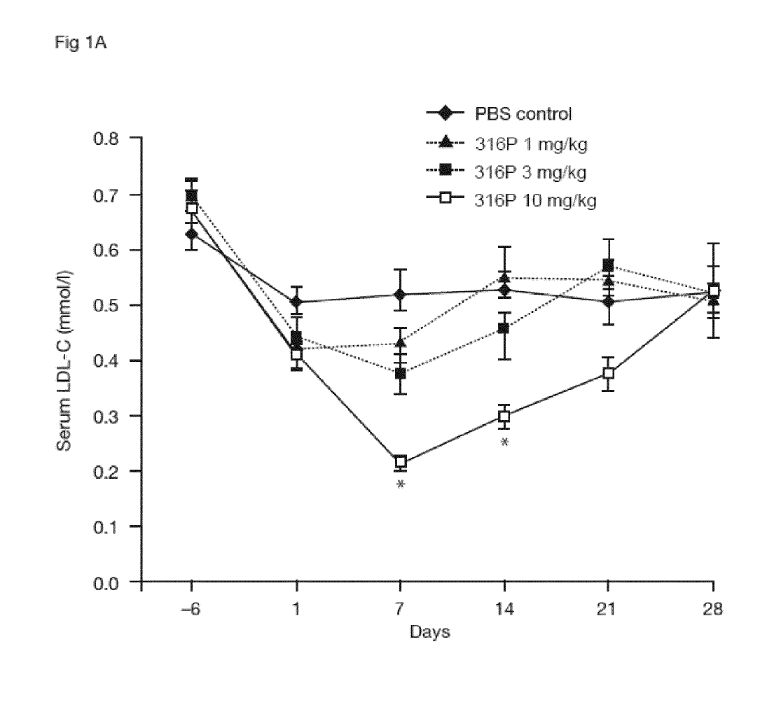

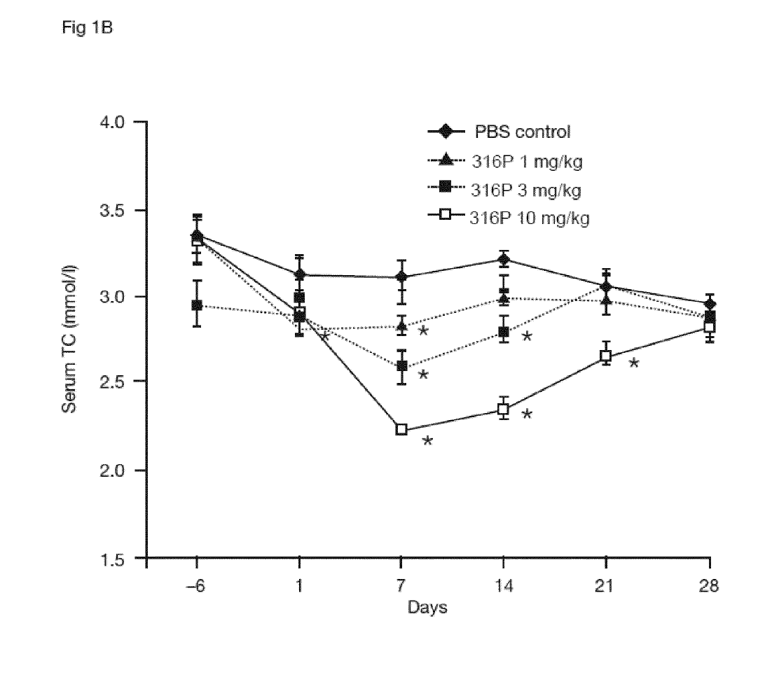

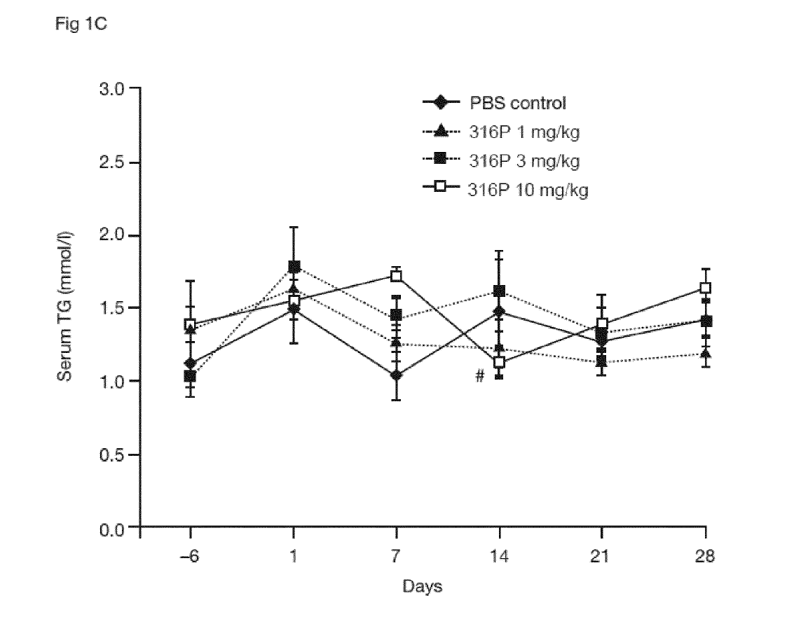

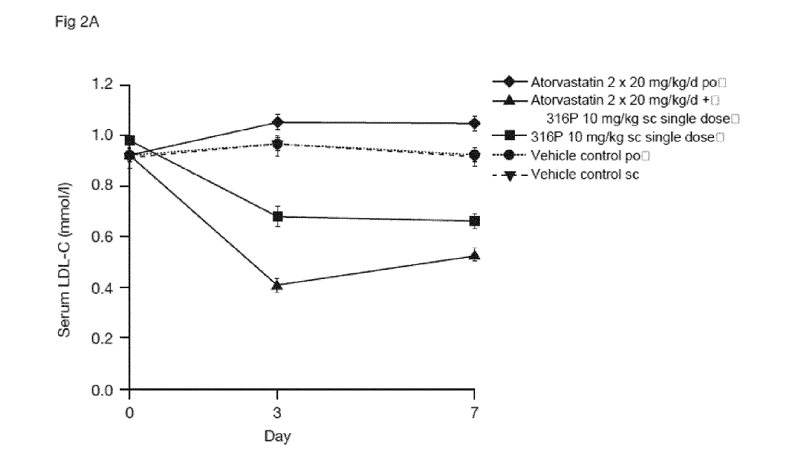

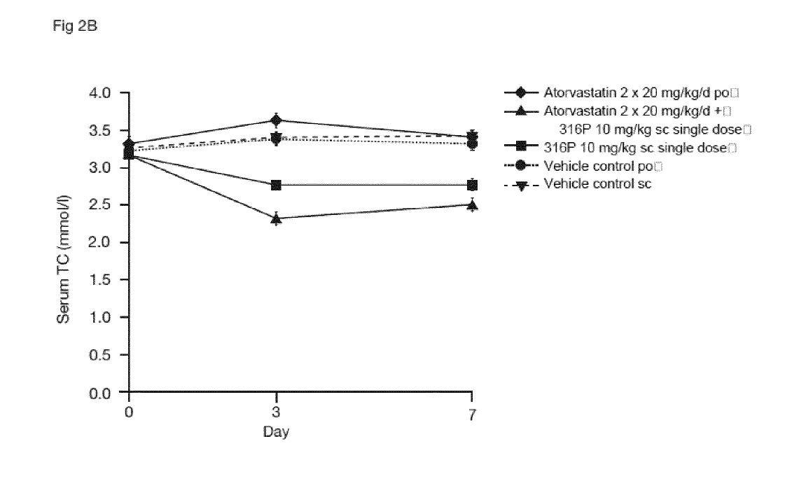

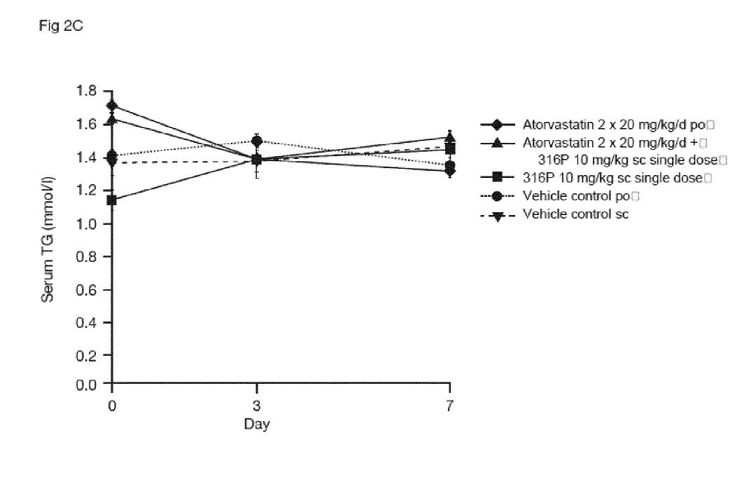

Lipid levels for each treatment group were comparable at baseline (−6 days for Study 1 and −7 days for Study 2) (FIGS. 1 and 2). Mean body weight gradually increased in all treatment groups, with no significant between-group differences in body weight, food consumption, or weight gain in either study. Compared with the PBS control groups, changes in serum TC, LDL-C, TG, HDL-C and phospholipid levels in the treatment-naive controls were not significantly different throughout the studies.

Study 1. Dose response to a single s.c. injection of 316P 1, 3, or 10 mg/kg in normolipidemic hamsters

A single s.c. injection of 316P 1, 3, or 10 mg/kg was associated with a dose-dependent decrease in LDL-C lasting more than 2 weeks (FIG. 1A). The maximal effect on LDL-C for the 1 mg/kg dose (17% reduction) was seen after Day 1 whereas the maximal effects for the 3 and 10 mg/kg doses (27% and 59% reductions, respectively) were observed after Day 7. The decrease in LDL-C was only statistically significant at Days 7 and 14 with the highest dose (10 mg/kg) (P 0.0014), after which LDL-C levels slowly increased. However, it should be noted that LDL-C levels did not reach PBS control-treated levels (0% reduction) until Day 28. The decrease in TC was significant for all given doses at Day 7 (9%, 17% and 28%, respectively; P 0.0325) and remained significant for the 2 higher doses at Day 14 (P 0.0011) and for the highest dose at Day 21 (P=0.0024) (FIG. 1B). The reduction in HDL-C was statistically significant but less pronounced than the effect on LDL-C (data not shown). Serum TG levels remained relatively constant for every dose throughout the study (FIG. 1C).

Analysis of serum 316P levels indicates that adequate and consistent drug doses were achieved across the respective groups (FIG. 3). Concentrations of serum 316P at the 24-hour time point appeared to be proportional to theoretical dosing levels of 1, 3, and 10 mg/kg. The highest level of 316P was detected after 1 day for all doses but began to decline within the following 6 days. A single s.c. injection of 316P 1, 3, or 10 mg/kg had no biologically relevant effects on safety parameters or on relative liver weight, hepatic cholesterol, TG, or phospholipid concentration (data not shown).

Study 2. Effects of atorvastatin 10 or 20 mg/kg twice daily with and without a single s.c. injection of 316P 10 mg/kg on lipid levels in normolipidemic hamsters

As in Study 1, a single s.c. injection of 316P 10 mg/kg had a potent effect on serum LDL-C and TC levels that was sustained until the study ended on Day 7 (FIGS. 2A, 2B). On its own, a twice-daily p.o. dose of atorvastatin 10 mg/kg or 20 mg/kg had no significant effect on serum LDL-C, TC (FIGS. 2A, B) or HDL-C after 7 days but reduced serum TG levels by 23% (FIG. 2C). However, in atorvastatin-treated animals, a single s.c. injection of 316P 10 mg/kg at Day 0 was associated with a significant decrease in serum LDL-C (46% with atorvastatin 2×10 mg/kg and 58% with atorvastatin 2×20 mg/kg), and TC (30% and 32%) at Day 3, an effect that was largely maintained at Day 7 (FIGS. 2A, 2B). Effects were similar irrespective of atorvastatin dose (FIGS. 2A, B). As in Study 1, the addition of 316P 10 mg/kg had no significant effect on TG levels beyond that achieved with atorvastatin 2×10 or 20 mg/kg alone (FIG. 2C). Again, the reduction in HDL-C was statistically significant but less pronounced than the effect on LDL-C (data not shown).

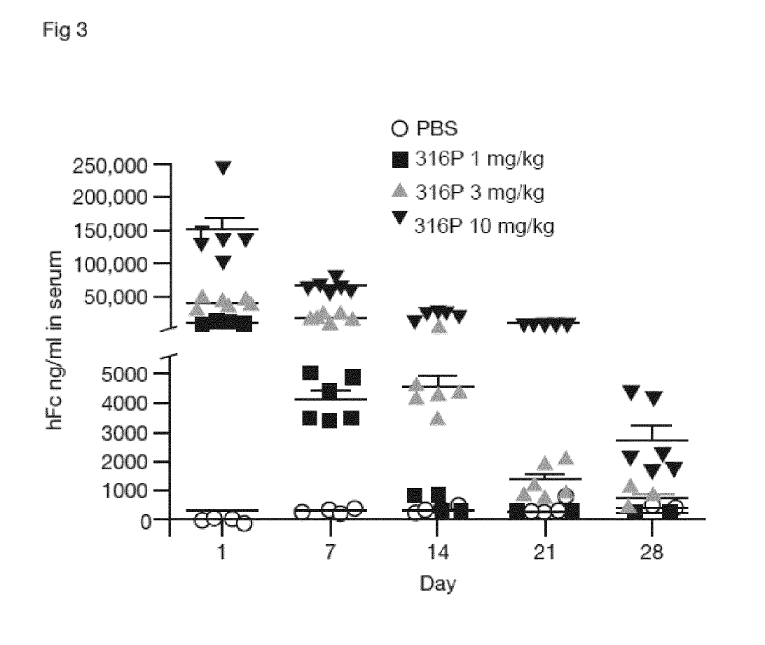

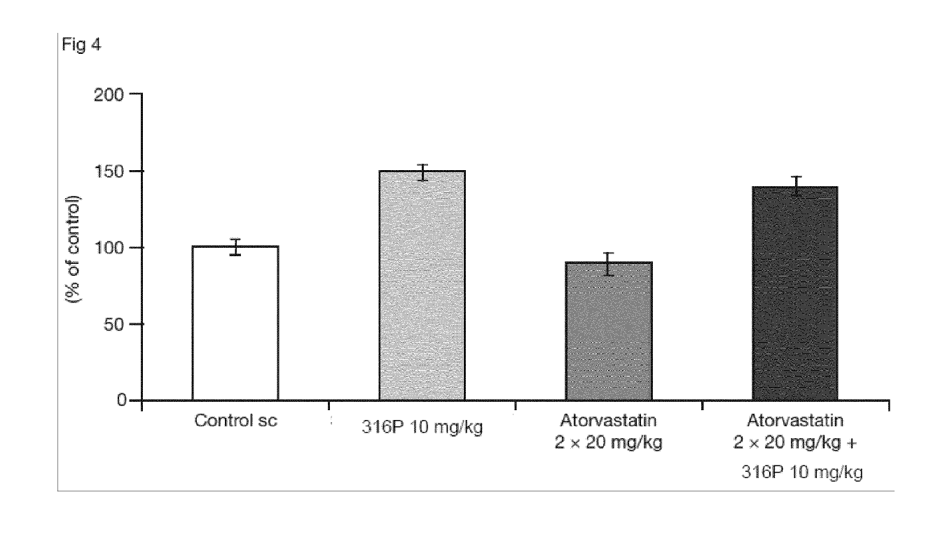

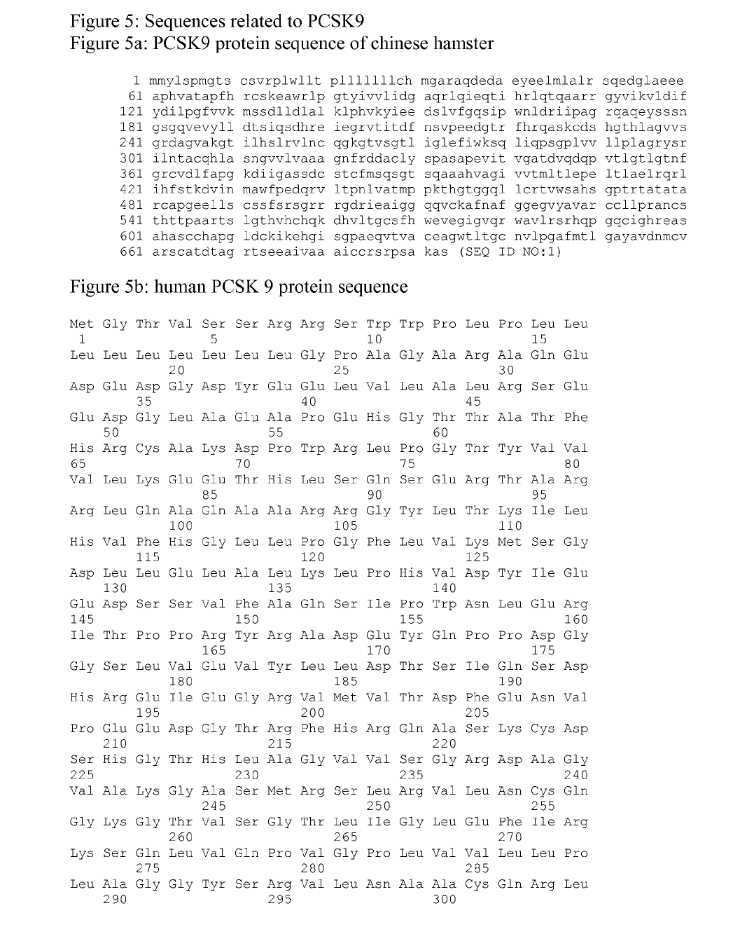

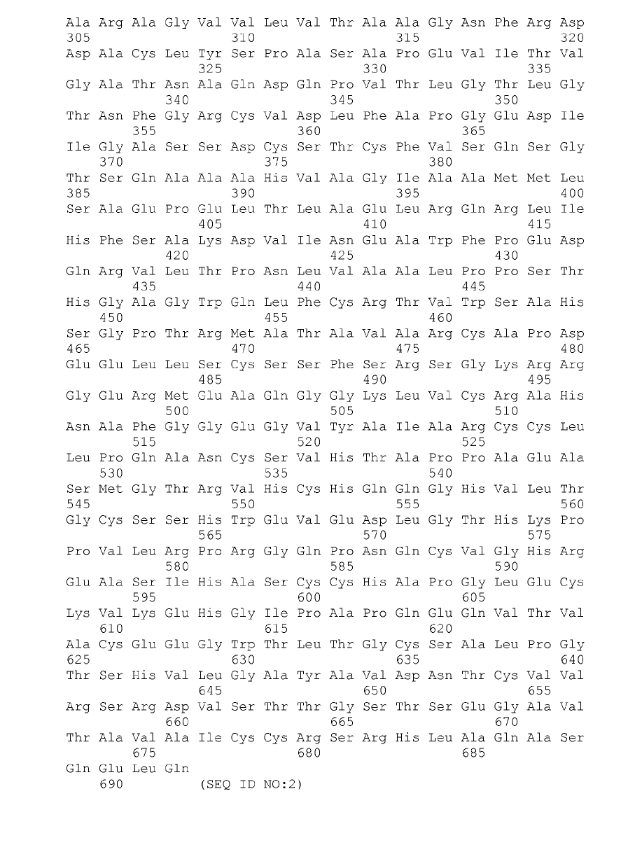

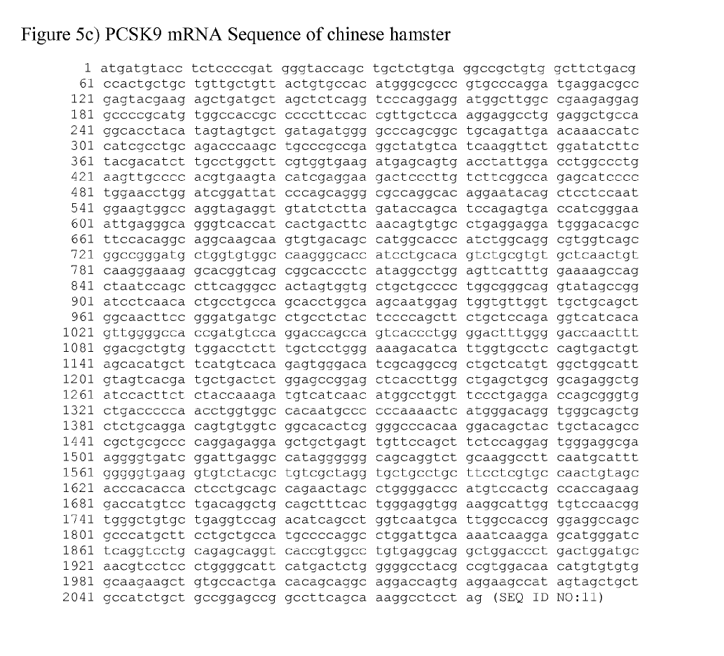

Compared with the PBS control groups, animals treated with 316P 10 mg/kg had the highest level of hepatic LDLR expression (149%), followed by those receiving 316P 10 mg/kg plus atorvastatin 2×20 mg/kg (140%) and then those receiving atorvastatin 2×20 mg/kg alone (89%) (FIG. 4). Whereas atorvastatin 2×10 or 20 mg/kg was associated with a slight, clinically insignificant increase in ALT levels, a single s.c. injection of 316P was well tolerated, with no relevant treatment-related effects on safety parameters and no clinically relevant effects on relative liver weight, hepatic cholesterol, TG or phospholipid concentration.

### Discussion

Previous studies have shown that 316P has a high binding affinity for PCSK9, both in humans and in a number of other species, and is able to antagonize PCSK9-mediated down-regulation of LDL uptake via LDLR, thereby reducing LDL-C. The present study showed that, in normolipidemic male Syrian hamsters, a single s.c. injection of 316P 1, 3, or 10 mg/kg resulted in a significant dose-dependent decrease in TC and LDL-C lasting more than 2 weeks, with a peak effect for the higher doses (3 and 10 mg/kg) at Day 7 (FIGS. 1A, 1B). The maximal 316P-mediated reduction in LDL-C observed in this study (59% at Day 7) is in accordance with an interim analysis of data from a human phase 1 study, in which LDL-C reduction exceeded 60% and lasted for 30 days following a single i.v. administration. No significant effects were observed on circulating TGs (FIGS. 1C, 2C), food intake or body weight with any dose in either study, but a significant reduction in HDL-C levels was observed in animals receiving the highest dose of 316P (10 mg/kg) (data not shown). Similar reductions in HDL-C have been observed in this animal model following treatment with statins. As humans do not show this effect with statin treatment, it is believed that the difference may be due to the relative abundance of apolipoprotein E in the HDL fractions of lower species compared with humans and their clearance by the up-regulated LDLR.

Pharmacokinetic data from Study 1 show that serum levels of 316P correlate with the dose-dependent effect on TC and LDL-C (FIGS. 1, 3), suggesting that the effects on lipid levels are due to PCSK9 inhibition. Moreover, Study 2 showed that a single s.c. injection of 316P 10 mg/kg led to a 1.5-fold effect in the level of hepatic LDLR compared with the PBS control (FIG. 4), suggesting that PCSK9 inhibition leads, in turn, to an increase in LDLR.

It has been well documented that rodents, including hamsters, are resistant to the LDL-C-lowering effects of statins but that statins effectively lower serum TG levels in these animals by inhibiting hepatic TG secretion. The discrepancy between the statin effects in rodents and those observed in humans is most likely due to differences in the expression of PCSK9. For example, a study in dyslipidemic hamsters treated with rosuvastatin showed that hepatic PCSK9 mRNA expression was induced to a greater extent than was LDLR mRNA. Further examination showed that rosuvastatin treatment was associated with increased levels of both SREBP-2 and hepatocyte nuclear factor 1 alpha (HNF1 alpha). Since SREBP-2 and HNF1 alpha both activate PCSK9 gene expression, whereas LDLR gene expression is only activated by SREBP-2, the net result was that hepatic LDLR levels were reduced and serum LDL-C levels were slightly increased. However, studies in human HepG2 or Huh7 cells showed that rosuvastatin induced a modest increase in LDLR (≈2-fold) and PCSK9 (≈3-fold) mRNA and protein but had no appreciable effect on the level of HNF1 alpha. These results suggest a species-specific regulation of PCSK9 via HNF1 alpha expression by statins. Consistent with these observations, the study showed that a twice-daily dose of atorvastatin 10 or 20 mg/kg had no significant effect on TC or LDL-C levels in normolipidemic Syrian hamsters, irrespective of dose, but that atorvastatin 2×20 mg/kg caused a 23% reduction in TG levels after 7 days of treatment (FIG. 2) and a 0.89-fold decrease in the level of hepatic LDLR (FIG. 4). Given that the mechanism for statin resistance in rodents is thought to involve an increased ratio of PCSK9:LDLR, the inhibition of PCSK9 activity should at least partially restore the lipid-lowering effect of statins in Syrian hamsters. To test this theory, Syrian hamsters received a single s.c. injection of 316P in combination with an ineffective dose of atorvastatin. As expected, serum LDL-C, TC, and HDL-C levels were significantly decreased compared with atorvastatin-treated animals (FIG. 2), an effect that was associated with a 1.4-fold increase in hepatic LDLR protein levels versus PBS-treated controls (FIG. 4). Moreover, the combination treatment was more effective for reducing serum LDL-C and TC levels than either of the monotherapies at any dose (FIG. 2).

With the exception of atorvastatin, which was associated with a biologically relevant increase in ALT, all treatments were well tolerated, with no clinically relevant changes in safety parameters or liver lipids. Together, these data suggest that a single s.c. injection of 316P dose dependently neutralizes PCSK9 activity and is safe and effective in overcoming the statin resistance observed in the hamster model. This study also confirms that the normolipidemic male Syrian hamster is a suitable model to investigate drugs targeting PCSK9.

### Further Preferred Aspects of the Invention

1. A method of testing the efficacy of a compound in modulating cholesterol levels in a subject, comprising:
   (a) measuring the total cholesterol level and/or LDL-C level and/or HDL-C level in a test sample of a test rodent obtained after administration of said compound to the test rodent,
   (b) measuring the total cholesterol level and/or LDL-C level and/or HDL-C level in a control sample obtained from a control rodent that has not been administered said compound,
   wherein the test rodent and the control rodent have decreased PCSK9 activity or expression, and
   (c) determining whether there is any difference in the total cholesterol levels and/or LDL-C levels and/or HDL-C levels of the test sample and the control sample, wherein the presence of any difference indicates that the compound is efficacious in modulating cholesterol levels in a subject.
2. The method according to aspect 1, wherein the rodent's decreased PCSK9 activity or expression is caused by a genomic knock-out of PCSK9, a stable or transient knock-down of PCSK9, or administration of a PCSK9 antagonist.
3. The method according to aspect 2, wherein the PCSK9 antagonist is an antibody or antigen-binding fragment of an antibody.
4. The method according to aspect 3, wherein the antibody or antibody binding fragment thereof comprises one or more of the sequences selected from the list consisting of SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 12, SEQ ID NO: 13, SEQ ID NO: 14, SEQ ID NO: 15, SEQ ID NO: 16, SEQ ID NO: 17, SEQ ID NO: 18 and SEQ ID NO: 19.
5. The method according to aspect 3, wherein the antibody or antigen-binding fragment of an antibody is administered to the rodent in a concentration of 1 mg/kg body weight, 3 mg/kg body weight, or 10 mg/kg body weight.
6. The method according to aspect 1, wherein the test rodent and the control rodent are the same, and the control sample is obtained prior to administration of the compound.
7. The method according to aspect 1, wherein the test rodent and the control rodent are different.
8. The method according to aspect 1, wherein the rodent is selected from the group consisting of hamster, mouse, rat, guinea pig, and rabbit.
9. The method according to aspect 8, wherein the rodent is a hamster.
10. The method according to aspect 9, wherein the rodent is a Syrian hamster.
11. The method according to aspect 1, wherein the rodent is a male rodent.
12. The method according to aspect 1, wherein the rodent is normolipidemic or hyperlipidemic.
13. The method according to aspect 1, wherein the compound is a biological molecule or a small molecule.
14. The method according to aspect 13, wherein the compound is a biological molecule selected from the group consisting of an antibody, an antisense molecule, an siRNA, and an aptamer.
15. The method according to aspect 13, wherein the compound is an HMG-CoA reductase inhibitor.
16. The method according to aspect 15, wherein the HMG-CoA reductase inhibitor is a statin.
17. The method according to aspect 1, wherein any difference in the total cholesterol levels and/or LDL-C levels and/or HDL-C levels of the test sample and the control sample is indicative of the same in vivo effect in other mammals such as humans, or in reptiles or birds.
18. The method according to aspect 1, wherein a decreased level of total cholesterol and/or a decreased level of LDL-C and/or an increased level of HDL-C level in the test sample compared to the control sample indicates that the compound is efficacious in the treatment or prevention of one or more diseases or disorders associated with elevated LDL-C levels in a subject.
19. The method according to aspect 1, wherein an increased level of total cholesterol and/or an increased level of LDL-C and/or a decreased level of HDL-C level in the test sample compared to the control sample indicates that the compound has adverse effects and may promote, contribute to or trigger of one or more of the diseases or disorders associated with elevated LDL-C levels in a subject.
20. The method according to aspect 17 or 18, wherein the disease or condition associated with elevated LDL-C levels is selected from the group consisting of hypercholesterolemia, hyperlipidemia, dyslipidemia, atherosclerosis and cardiovascular diseases.
21. The method according to aspect 1, wherein the control rodent is from the same species as the test rodent.
22. The method according to aspect 20, wherein the control rodent is from the same strain as the test rodent.
23. The method according to aspect 1, wherein the total cholesterol level and/or LDL-C level and/or HDL-C level is determined by means of a colorimetric, photometric, fluorometric gravimetric or spectroscopic method.
24. The method according to aspect 1, wherein the test sample and the control sample are blood, plasma or serum.
25. A rodent for use in identifying a drug for the treatment of a disease associated with elevated cholesterol levels and preferably associated with elevated LDL-C levels, wherein the rodent has decreased PCSK9 levels in comparison to a control rodent.
26. The rodent according to aspect 25, wherein the rodent's decreased PCSK9 activity or expression is caused by a genomic knock-out of PCSK9, a stable or transient knock-down of PCSK9, or administration of a PCSK9 antagonist.
27. The rodent according to aspect 26, wherein the PCSK9 antagonist is an antibody or antigen-binding fragment of an antibody.
28. The rodent according to aspect 27, wherein the antibody or antibody binding fragment thereof comprises one or more of the sequences selected from the list consisting of SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10 SEQ ID NO: 12, SEQ ID NO: 13, SEQ ID NO: 14, SEQ ID NO: 15, SEQ ID NO: 16, SEQ ID NO: 17, SEQ ID NO: 18 and SEQ ID NO: 19
29. The rodent according to aspect 28, wherein the antibody or antigen-binding fragment of an antibody is administered to the rodent in a concentration of 1 mg/kg body weight, 3 mg/kg body weight, or 10 mg/kg body weight.
30. The rodent according to aspect 26, wherein the rodent is selected from the group consisting of hamster, mouse, rat, guinea pig, and rabbit.
31. The rodent according to aspect 30, wherein the rodent is a hamster.
32. The rodent according to aspect 31, wherein the rodent is a Syrian hamster.
33. The rodent according to aspect 26, wherein the rodent is a male rodent.
34. The rodent according to aspect 26, wherein the rodent is normolipidemic or hyperlipidemic.
35. A method for the preparation of a rodent suitable for use as a model system for determining the cholesterol-modulating effect of a drug, comprising providing a rodent or a blastocyst of a rodent and lowering its PCSK9 level by a method selected from the group consisting of a genomic knock-out of PCSK9, a stable knock-down of PCSK9, transient knock-down of PCSK9, and administration of a PCSK9 antagonist.
36. A method of testing the efficacy of an antibody or an antigen-binding fragment which specifically binds PCSK9 for the treatment of a disease or condition associated with elevated LDL-C levels, comprising:
   (a) determining the total cholesterol level and/or LDL-C level and/or HDL-C level in a test sample of a test rodent obtained after administration of said antibody or antigen-binding fragment thereof to the test rodent,
   (b) determining the total cholesterol level and/or LDL-C level and/or HDL-C level in a control sample obtained from a control rodent that has not been treated with said antibody or antigen-binding fragment thereof, and
   (c) determining whether there is any difference in the total cholesterol levels and/or LDL-C levels and/or HDL-C levels of the test sample and the control sample,
   wherein the antibody is efficacious for the treatment of the disease or condition if
      (i) the total cholesterol level and/or LDL-C level is lower in the test sample than in the control sample, and/or
      (ii) the HDL-C level is higher in the test sample than in the control sample;
   and wherein the antibody is has an adverse effect on the disease or condition if the total cholesterol level and/or LDL-C level is higher in the test sample than in the control sample.
37. The method according to aspect 36, wherein the antibody or antigen-binding fragment of an antibody is administered to the rodent in a concentration of 1 mg/kg body weight, 3 mg/kg body weight, or 10 mg/kg body weight.
38. The method according to aspect 36, wherein the test rodent and the control rodent are the same, and the control sample is obtained prior to administration of the antibody or antigen-binding fragment.
39. The method according to aspect 34, wherein the test rodent and the control rodent are different.
38. The method according to aspect 36, wherein the rodent is selected from the group consisting of hamster, mouse, rat, guinea pig, and rabbit.
41. The method according to aspect 40, wherein the rodent is a hamster.
42. The method according to aspect 41, wherein the rodent is a Syrian hamster.
43. The method according to aspect 43, wherein the rodent is a male rodent.
44. The method according to aspect 36, wherein the rodent is normolipidemic or hyperlipidemic.
45. The method according to aspect 36, wherein any difference in the total cholesterol levels and/or LDL-C levels and/or HDL-C levels of the test sample and the control sample is indicative of the same in vivo effect in other mammals such as humans, or in reptiles or birds.
46. The method according to aspect 36, wherein the disease or condition associated with elevated LDL-C levels is selected from the group consisting of hypercholesterolemia, hyperlipidemia, dyslipidemia, atherosclerosis and cardiovascular diseases.
47. The method according to aspect 36, wherein the control rodent is from the same species as the test rodent.
48. The method according to aspect 36, wherein the control rodent is from the same strain as the test rodent.
49. The method according to aspect 36, wherein the total cholesterol level and/or LDL-C level and/or HDL-C level is determined by means of a colorimetric, photometric, fluorometric gravimetric or spectroscopic method.
50. The method according to aspect 36, wherein the test sample and the control sample are blood, plasma or serum.
51. A kit for testing the efficacy of a compound in modulating cholesterol levels in a subject, comprising: a rodent and a PCSK 9 specific antagonist.
52. The kit of aspect 51, wherein the PCSK9 specific antagonist is an antibody or antigen-binding fragment of an antibody.
53. The kit of aspect 52, wherein the antibody or antibody binding fragment thereof comprises one or more of the sequences selected from the list consisting of SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 12, SEQ ID NO: 13, SEQ ID NO: 14, SEQ ID NO: 15, SEQ ID NO: 16, SEQ ID NO: 17, SEQ ID NO: 18 and SEQ ID NO: 19
54. The kit according to aspect 51, wherein the rodent is selected from the group consisting of hamster, mouse, rat, guinea pig, and rabbit.
55. The kit according to aspect 54, wherein the rodent is a hamster.
56. The kit according to aspect 55, wherein the rodent is a Syrian hamster.
57. The kit according to aspect 51, wherein the rodent is a male rodent.
58. The kit according to aspect 51, wherein the rodent is normolipidemic or hyperlipidemic.

1. A method for screening compounds to identify therapeutic candidates for the modulation of a disease or condition associated with elevated LDL-C levels, said method comprising:
(a) providing a rodent
(b) administering a test compound to the rodent and
(c) detecting whether said compound increases or decreases one or more parameters selected from the group consisting of: total cholesterol (TC), low-density cholesterol (LDL-C) and high-density cholesterol (HDL-C) in said rodent in comparison to a control rodent;
wherein a modulation of one or more of said parameters indicates that said compound is a candidate for modulating said disease or condition in vivo.

2. A method for screening compounds to identify therapeutic candidates for the modulation of a disease or condition associated with elevated LDL-C level, said method comprising:
(a) providing a rodent
(b) administering a test compound to the rodent
(c) determining one or more parameters of the rodent selected from the group consisting of: total cholesterol (TC), low-density cholesterol (LDL-C) and high-density cholesterol (HDL-C) before treatment of the rodent with the compound
(d) determining the one or more parameters after treatment of the rodent with the compound, and
(e) comparing the results obtained in (a) with those obtained in (b),
wherein a difference of the parameters of (a) in comparison with those of (b) indicates that the compound is a candidate for modulating said disease or condition in vivo.

3. Method according to claim 1 or 2, wherein the parameters are determined in vitro in one or more taken samples of the rodent or rodents.

4. In vitro method according to claim 3 comprising steps (c), of claim 1 or comprising steps (c), (d) and (e) of claim 2.

5. Method according to one of the claims 1 to 4, wherein the modulation of the one or more parameters is indicative of the same in vivo effect in other mammals such as humans, or in reptiles or birds.

6. Method according to one of the claims 1 to 5, wherein
a decrease of total cholesterol and/or of LDL-C and/or increase of HDL-C is indicative that said compound is a candidate for treating or preventing one or more of said diseases or conditions in vivo, and wherein
an increase of total cholesterol and/or of LDL-C is indicative that said compound exhibits adverse effects and is a candidate for promoting or inducing one or more of said diseases or conditions in vivo.

7. Method according to one of the claims 1 to 6, wherein the compound is a biological molecule, such as an antibody, antisense, siRNA or aptamer or a small molecule such as an HMG-CoA reductase inhibitor, e.g. a statin.

8. Method according to one of the claims 1 or 2, wherein the rodents of claim 1 or the rodent of claim 2 has decreased PCSK9 levels or activity in comparison to a reference.

9. Method according to one of the claims 1, 2 or 8, wherein an antagonist of PCSK9 and preferably an antibody specifically binding to PCSK9 has been administered to the rodents of claim 1 prior to step (c) or to the rodent of claim 3 prior to step (a).

10. Method according to claim 9, wherein the compound is an inhibitor of HMG-CoA reductase and preferably a statin.

11. A method of testing the efficacy of an antibody or an antigen-binding fragment thereof which specifically binds hPCSK9 for the treatment of a disease or condition associated with elevated LDL-C levels, said method comprising:
(a) administering said antibody to a rodent; and
(b) determining the total cholesterol, LDL-C or HDL-C level of the rodent before and after administration of said antibody or antigen-binding fragment thereof to the rodent,
wherein a reduction of the total cholesterol and/or LDL-C level and/or a increase of the HDL-C level determined after administration of the antibody relative to the predose level determined before administration of the antibody is indicative that the antibody or antigen-binding fragment thereof is efficacious for the treatment of said disease or condition, and
wherein the increase of the total cholesterol level and/or the LDL-C level determined after administration of the antibody relative to the predose level determined before administration of the antibody is indicative that the antibody exhibits adverse effects in promoting, contributing to or triggering said disease or condition in vivo.

12. A method of testing the efficacy of an antibody or an antigen-binding fragment thereof which specifically binds hPCSK9 for the modulation of a disease or condition associated with elevated LDL-C levels, said method comprising:
(a) determining the total cholesterol level, the LDL-C level and/or the HDL-level in an in vitro sample obtained from a rodent before treatment of the rodent with the antibody,
(b) determining the total cholesterol level, the LDL-C level and/or the HDL-level in an in vitro sample obtained from the rodent after treatment of the rodent with the antibody, and
(c) comparing the results obtained in (a) with those obtained in (b),
wherein a reduction of the total cholesterol and/or LDL-C level and/or a increase of the HDL-C level determined in (b) relative to the predose level determined in (a) before administration of the antibody is indicative that the antibody or antigen-binding fragment thereof is efficacious for the treatment and/or prevention of said disease or condition, and
wherein the increase of the total cholesterol level and/or the LDL-C level in (b) in comparison the predose level detected in (a) is indicative that the antibody exhibits adverse effects in promoting, contributing to or triggering said disease or condition in vivo.

13. A method according to claim 11 or 12, wherein the rodent has been administered a compound lowering total cholesterol and/or LDL-C levels and/or increasing HDL-C levels in humans and wherein the compound has been administered prior to determining of the predose level in the method of claim 11 and prior to the taking of the sample in step (a) of claim 12.

14. A method of testing the efficacy of an antibody or an antigen-binding fragment thereof which specifically binds hPCSK9 for the treatment of a disease or condition associated with elevated LDL-C levels, said method comprising:
(a) administering said antibody to a rodent; and
(b) determining the efficacy of said antibody or antigen-binding fragment thereof by determining the total cholesterol level and/or LDL-C level and/or HDL-C level of the rodent after administration of said antibody or antigen-binding fragment thereof,
(c) determining the total cholesterol level and/or LDL-C level and/or HDL-C level of a control rodent that has not been treated with the antibody and has preferably obtained a placebo,
wherein the antibody is considered efficacious for the treatment of the disease or condition if the total cholesterol level and/or LDL-C level determined in (c) is lower and/or the HDL-C level determined in (c) is higher than that determined in (b) and
wherein the antibody is considered to exhibit adverse effects if the total cholesterol level and/or LDL-C level determined in (c) is higher than that determined in (b).

15. An in vitro method of testing the efficacy of an antibody or an antigen-binding fragment thereof which specifically binds hPCSK9 for the treatment of a disease or condition associated with elevated LDL-C levels, said method comprising:
(a) determining the total cholesterol level and/or LDL-C level and/or HDL-C level in a sample of a rodent obtained after administration of said antibody or antigen-binding fragment thereof to the rodent,
(b) determining the total cholesterol level and/or LDL-C level and/or HDL-C level in a control sample obtained from a rodent that has not been treated with said antibody or antigen-binding fragment thereof,
wherein the antibody is considered efficacious for the treatment of the disease or condition if the total cholesterol level and/or LDL-C level determined in (b) is lower and/or the HDL-C level determined in (b) is higher than that determined in (a) and
wherein the antibody is considered to exhibit adverse effects if the total cholesterol level and/or LDL-C level determined in (b) is higher than that determined in (a).

16. A method according to claim 14 or 15, wherein the rodent and the control rodent have been administered a compound lowering total cholesterol and/or LDL-C and/or increase HDL-C in humans.

17. A method according to one of the claims 11 or 16, wherein the compound is an HMG-CoA reductase inhibitor and preferably a statin.

18. A method for testing the efficacy of a compound in modulating cholesterol levels in a subject, comprising the steps:
(a) providing a rodent;
(b) administering an antibody or an antigen-binding fragment thereof which specifically binds PCSK9 to the rodent;
(c) administering a test compound to said rodent;
(d) determining one or more parameters of the rodent selected from the group consisting of: the total cholesterol level, LDL-C level or HDL-C level, after administration of the test compound,
(e) determining the same one or more parameter(s) of a control rodent that has not been challenged with the test compound
wherein a difference in the cholesterol (total or LDL-C or HDL-C) determined in (a) and determined in (b) indicates that the test compound is efficacious in modulating cholesterol levels in a subject.

19. An in vitro method for testing the efficacy of a compound in modulating cholesterol levels in a subject, comprising the steps:
(a) determining in a sample of a rodent taken after the rodent has been applied a test compound one or more of the parameters selected from the group consisting of: the total cholesterol level, LDL-C level or HDL-C level,
(b) determining the same one or more parameter(s) in a sample of a control rodent that has not been challenged with the test compound
wherein both animals have been administered an antibody or an antigen-binding fragment thereof which specifically binds PCSK9 in addition to the test compound, and
wherein a difference in the cholesterol (total C and/or LDL-C and/or HDL-C) determined in (a) and determined in (b) indicates that the test compound is efficacious in modulating cholesterol levels in a subject.

20. Method according to one of the claims 18 or 19, wherein
a decreased level of cholesterol (total and/or LDL-C) and/or an increased level of HDL-C determined in the rodent or in a sample thereof as compared to the total, LDL or HDL cholesterol level in the control rodent indicates that the test compound is efficacious in the treatment or prevention of one or more of the diseases or disorders associated with elevated LDL-C levels in a subject, and wherein
an increased level of cholesterol (total and/or LDL-C) determined in the test rodent or in a sample thereof as compared to the total- or LDL cholesterol level in the control rodent indicates that the test compound has adverse effects and may promote, contribute to or trigger a disease or condition associated with elevated LDL-C levels.

21. A method for testing the efficacy of a compound in modulating cholesterol levels in a subject, comprising the steps:
(a) providing a rodent;
(b) administering an antibody or an antigen-binding fragment thereof which specifically binds PCSK9 to the rodent;
(c) administering a test compound to said rodent;
(d) determining in the rodent one or more of the parameters selected from the group consisting of: total cholesterol levels, LDL-C levels or HDL-C levels
(i) before administration of the test compound to the rodent and
(ii) after administration of the test compound to the rodent:,
(e) comparing the parameters obtained in (d)(ii) and (d)(ii)
wherein a difference in the parameters obtained in (d) (ii) with the parameter obtained in (d) (ii) indicates that the test compound compound is efficacious in modulating cholesterol levels in a subject.

22. An in vitro method for testing the efficacy of a compound in modulating cholesterol levels in a subject, comprising the steps:
(a) determining one or more of the parameters selected from the group consisting of: total cholesterol levels, LDL-C levels or HDL-C levels,
(i) in a sample of a rodent obtained before administration of the test compound to the rodent, and
(ii) in a sample of the same rodent obtained administration of the test compound, and
(b) comparing the parameters determined in (d)(ii) and (d)(ii)
wherein the rodent has been administered an antibody or an antigen-binding fragment thereof which specifically binds PCSK9 in conjunction with the test administration compound and
wherein a difference in the parameters obtained in (d) (ii) with the parameter obtained in (d) (ii) indicates that the test compound compound is efficacious in modulating cholesterol levels in a subject.

23. Method according to claim 21 or 22, wherein a decreased level of cholesterol (total or LDL-C) and/or an increased level increase of the HDL-C level in (ii) in comparison to (i) indicates that the test compound is efficacious in the treatment or prevention of one or more of the diseases or disorders associated with elevated LDL-C levels in a subject.

24. Method according to claim 21 or 22, wherein an increased level of cholesterol (total or LDL-C) in (ii) in comparison to (i) indicates that the test compound has adverse effects and may promote, contribute to or trigger of one or more of the diseases or disorders associated with elevated LDL-C levels in a subject.

25. Method according to one of the claims 20 to 24, wherein the control rodent is from the same species and preferably also from the same strain as the test rodent.

26. Method according to one of the claims 1, w, 18, 19, 21 or 22, wherein the test compound is a PCSK9-inhibitor, such as a PCSK9 antibody or an HMG-CoA reductase inhibitor, e.g. a statin.

27. Method according to one of the claims 9, 11, 12, 14, 15, 18, 19, 21, 22, or 26, wherein the antibody is administered to the rodent in a concentration of 1 mg/kg body weight, 3 mg/kg body weight, or 10 mg/kg body weight.

28. Method according to one of the claims 11, 14, 18 or 21, wherein the parameter or level of cholesterol is determined in a taken sample.

29. Method according to one of the claims 3, 4, 12, 15, 19, 22 or 28, wherein the cholesterol level is determined by means of a colorimetric, photometric, fluorometric gravimetric or spectroscopic method.

30. Method according to one of the claims 3, 4, 12, 15, 19, 22, 28 or 29, wherein the sample is blood, plasma or serum.

31. Method according to one of the claims 1 to 30, wherein the result of the method is interpreted to be indicative for other species than the species used in the method, such as other rodents than the used species, other mammals than the used species and preferably humans.

32. Method according to one of the claims 1 to 31, wherein the disease or condition associated with elevated LDL-C levels is selected from the group consisting of hypercholesterolemia, hyperlipidemia, dyslipidemia, atherosclerosis and cardiovascular diseases.

33. Rodent for use in identifying a drug for the treatment of a disease associated with elevated cholesterol levels and preferably associated with elevated LDL-C levels, wherein the rodent has decreased PCSK9 levels in comparison to a control rodent.

34. Use of a rodent, with decreased PCSK9 levels in comparison to a control rodent as model system for determining the cholesterol-modulating effect and preferably of the cholesterol-lowering effect of a drug.

35. Rodent according to claim 33 or use according to claim 34, wherein the drug is an HMG-CoA reductase inhibitor.

36. Rodent according to claim 33 for use in a method according to one of the claims 1, 2, 18, 19, 21 or 22.

37. Rodent according to claim 35 or 36 or use according to claim 34, wherein the lowered PCSk-9 activity or expression level is caused by a genomic knock-out of PCSK9, a stable or transient knock-down of PCSK9 or administration of a PCSK-9 antagonist.

38. Method for the preparation of a rodent suitable for use as model system for determining the cholesterol-modulating effect and preferably of the cholesterol-lowering effect of a drug, the method comprising providing a rodent or a blastocyst of a rodent and lowering its PCSK9 level by means of a genomic knock-out of PCSK9, a stable or transient knock-down of PCSK9 or administration of a PCSK-9 antagonist.

39. Rodent or use according to claim 37, or method according to claim 39, wherein the lowered PCSK-9 activity or expression level is caused by administration of a PCSK9 antagonist, preferably a specific PCSK-9 antibody to the rodent.

40. Method according to one of the claims 1 to 32, use according to claim 34 or rodent according to one of the claims 33 or 39, wherein the rodent is selected from hamster, mouse, rat, guinea pig and rabbit and is preferably a hamster.

41. Method, use or rodent according to claim 40, wherein the rodent is a hamster and preferably a syrian hamster.

42. Method, use or rodent according to one of the claims 1 to 41, wherein the rodent is a male rodent.

43. Method, use or rodent according to one of the claims 1 to 42, wherein the rodent is normolipidemic or hyperlipidemic and preferably normolipidemic.

44. Rodent, preferably hamster, obtained by a method according to one of the claims 39 to 43, and preferably obtained by administration of a PCSK-9 specific antibody.

45. Kit for a method according to one of the claims 1 to 32 comprising a rodent, preferably a hamster and a PCSK 9 specific antagonist, such as a PCSK9 specific antibody and optionally comprising one or more of the further components according to one of the claims 44 to 47.

46. An article of manufacture comprising
(a) a packaging material or container;
(b) an antibody or an antigen-binding fragment thereof which specifically binds hPCSK9; and
(c) a data carrier such as a label or packaging insert contained within the packaging material containing instructions for carrying out a method according to one of the claims 1 to 38 for profiling or identifying compounds for use in the treatment or prevention of hypercholesterolemia, hyperlipidemia, dyslipidemia, atherosclerosis and cardiovascular diseases and optionally
(d) one or more buffers and/or reagents for determining total cholesterol levels, LDL-C levels or HDL-C levels in a sample.

47. An article of manufacture comprising
(a) a packaging material or container;
(b) reagents and buffers for determining total cholesterol levels, LDL-C levels or HDL-C levels in a sample; and
(c) a data carrier such as a label containing instructions for carrying out a method according to one of the claims 1 to 38 and optionally
(d) an antibody or an antigen-binding fragment thereof which specifically binds hPCSK9.

48. An article of manufacture according to one of the claims 44 or 45 further comprising one or more rodents.

49. An article of manufacture according to one of the claims 44 to 46, comprising
a data carrier, wherein the data carrier comprises information such as
(i) instructions for use of the antibody or fragment thereof
(ii) quality information such as information about the lot/batch number of the antibody or of the article of, the manufacturing or assembly site or the expiry or sell-by date, information concerning the correct storage or handling of the article,
(iii) information concerning the composition of the buffer(s), diluent(s), reagent(s) for determining the cholesterol levels or for use of the antibody,
(iv) information concerning the interpretation of information obtained when performing the above-mentioned methods,
(v) a warning concerning possible misinterpretations or wrong results when applying unsuitable methods, and/or
(vi) a warning concerning possible misinterpretations or wrong results when using unsuitable reagent(s) and/or buffer(s).

50. Method according to one of the claims 1 to 32, use according to one of the claims 34 to 39, rodent according to one of the claims 35 to 39, kit according to claim 43 or article of manufacture according to one of the claims 44 to 47, wherein the rodent is selected from hamster, mouse, rat, guinea pig and rabbit and is preferably a hamster.

51. Method, use, rodent, kit or article of manufacture according to claim 48, wherein the rodent is a hamster and preferably a syrian hamster.

52. Method, use, rodent, kit or article of manufacture according to one of the claims 1 to 49, wherein the rodent is normolipidemic or hyperlipidemic and preferably normolipidemic.

53. Method, use, rodent, kit or article of manufacture according to claim 50, wherein the rodent is a normolipidemic syrian hamster, and preferably a normolipidemic male syrian hamster.


SEQUENCE LISTING (19 sequences)

SEQ ID NO: 1 - protein, 693 aa, Hamster
MMYLSPMGTSCSVRPLWLLTPLLLLLLLCHMGARAQDEDAEYEELMLALRSQEDGLAEEEAPHVATAPFHRCSKEAWRLPGTYIVVLIDGAQRLQIEQTIHRLQTQAARRGYVIKVLDIFYDILPGFVVKMSSDLLDLALKLPHVKYIEEDSLVFGQSIPWNLDRIIPAGRQAQEYSSSNGSGQVEVYLLDTSIQSDHREIEGRVTITDFNSVPEEDGTRFHRQASKCDSHGTHLAGVVSGRDAGVAKGTILHSLRVLNCQGKGTVSGTLIGLEFIWKSQLIQPSGPLVVLLPLAGRYSRILNTACQHLASNGVVLVAAAGNFRDDACLYSPASAPEVITVGATDVQDQPVTLGTLGTNFGRCVDLFAPGKDIIGASSDCSTCFMSQSGTSQAAAHVAGIVVTMLTLEPELTLAELRQRLIHFSTKDVINMAWFPEDQRVLTPNLVATMPPKTHGTGGQLLCRTVWSAHSGPTRTATATARCAPGEELLSCSSFSRSGRRRGDRIEAIGGQQVCKAFNAFGGEGVYAVARCCLLPRANCSTHTTPAARTSLGTHVHCHQKDHVLTGCSFHWEVEGIGVQRWAVLRSRHQPGQCIGHREASAHASCCHAPGLDCKIKEHGISGPAEQVTVACEAGWTLTGCNVLPGAFMTLGAYAVDNMCVARSCATDTAGRTSEEAIVAAAICCRSRPSAKAS

SEQ ID NO: 2 - protein, 692 aa, Homo sapiens
MGTVSSRRSWWPLPLLLLLLLLLGPAGARAQEDEDGDYEELVLALRSEEDGLAEAPEHGTTATFHRCAKDPWRLPGTYVVVLKEETHLSQSERTARRLQAQAARRGYLTKILHVFHGLLPGFLVKMSGDLLELALKLPHVDYIEEDSSVFAQSIPWNLERITPPRYRADEYQPPDGGSLVEVYLLDTSIQSDHREIEGRVMVTDFENVPEEDGTRFHRQASKCDSHGTHLAGVVSGRDAGVAKGASMRSLRVLNCQGKGTVSGTLIGLEFIRKSQLVQPVGPLVVLLPLAGGYSRVLNAACQRLARAGVVLVTAAGNFRDDACLYSPASAPEVITVGATNAQDQPVTLGTLGTNFGRCVDLFAPGEDIIGASSDCSTCFVSQSGTSQAAAHVAGIAAMMLSAEPELTLAELRQRLIHFSAKDVINEAWFPEDQRVLTPNLVAALPPSTHGAGWQLFCRTVWSAHSGPTRMATAVARCAPDEELLSCSSFSRSGKRRGERMEAQGGKLVCRAHNAFGGEGVYAIARCCLLPQANCSVHTAPPAEASMGTRVHCHQQGHVLTGCSSHWEVEDLGTHKPPVLRPRGQPNQCVGHREASIHASCCHAPGLECKVKEHGIPAPQEQVTVACEEGWTLTGCSALPGTSHVLGAYAVDNTCVVRSRDVSTTGSTSEGAVTAVAICCRSRHLAQASQELQ

SEQ ID NO: 3 - protein, 8 aa, Artificial sequences
  Antibody sequence
GFTFNNYA

SEQ ID NO: 4 - protein, 8 aa, Artificial sequences
  Antibody sequence
ISGSGGTT

SEQ ID NO: 5 - protein, 11 aa, Artificial sequences
  Antibody sequence
AKDSNWGNFDL

SEQ ID NO: 6 - protein, 12 aa, Artificial sequences
  Antibody sequence
QSVLYRSNNRNF

SEQ ID NO: 7 - protein, 3 aa, Artificial sequences
  Antibody sequence
WAS

SEQ ID NO: 8 - protein, 9 aa, Artificial sequences
  Antibody sequence
QQYYTTPYT

SEQ ID NO: 9 - protein, 118 aa, Artificial sequences [sequence omitted]

SEQ ID NO: 10 - protein, 113 aa, Artificial sequences [sequence omitted]

SEQ ID NO: 11 - DNA, 2082 bp, Hamster
atgatgtacctctccccgatgggtaccagctgctctgtgaggccgctgtggcttctgacgccactgctgctgttgctgttactgtgccacatgggcgcccgtgcccaggatgaggacgccgagtacgaagagctgatgctagctctcaggtcccaggaggatggcttggccgaagaggaggccccgcatgtggccaccgcccccttccaccgttgctccaaggaggcctggaggctgccaggcacctacatagtagtgctgatagatggggcccagcggctgcagattgaacaaaccatccatcgcctgcagacccaagctgcccgccgaggctatgtcatcaaggttctggatatcttctacgacatcttgcctggcttcgtggtgaagatgagcagtgacctattggacctggccctgaagttgccccacgtgaagtacatcgaggaagactcccttgtcttcggccagagcatcccctggaacctggatcggattatcccagcagggcgccaggcacaggaatacagctcctccaatggaagtggccaggtagaggtgtatctcttagataccagcatccagagtgaccatcgggaaattgagggcagggtcaccatcactgacttcaacagtgtgcctgaggaggatgggacacgcttccacaggcaggcaagcaagtgtgacagccatggcacccatctggcaggcgtggtcagcggccgggatgctggtgtggccaagggcaccatcctgcacagtctgcgtgtgctcaactgtcaagggaaaggcacggtcagcggcaccctcataggcctggagttcatttggaaaagccagctaatccagccttcagggccactagtggtgctgctgcccctggcgggcaggtatagccggatcctcaacactgcctgccagcacctggcaagcaatggagtggtgttggttgctgcagctggcaacttccgggatgatgcctgcctctactccccagcttctgctccagaggtcatcacagttggggccaccgatgtccaggaccagccagtcaccctggggactttggggaccaactttggacgctgtgtggacctctttgctcctgggaaagacatcattggtgcctccagtgactgtagcacatgcttcatgtcacagagtgggacatcgcaggccgctgctcatgtggctggcattgtagtcacgatgctgactctggagccggagctcaccttggctgagctgcggcagaggctgatccacttctctaccaaagatgtcatcaacatggcctggttccctgaggaccagcgggtgctgacccccaacctggtggccacaatgccccccaaaactcatgggacaggtgggcagctgctctgcaggacagtgtggtcggcacactcggggcccacaaggacagctactgctacagcccgctgcgccccaggagaggagctgctgagttgttccagcttctccaggagtgggaggcgaaggggtgatcggattgaggccataggggggcagcaggtctgcaaggccttcaatgcatttgggggtgaaggtgtctacgctgtcgctaggtgctgcctgcttcctcgtgccaactgtagcacccacaccactcctgcagccagaactagcctggggacccatgtccactgccaccagaaggaccatgtcctgacaggctgcagctttcactgggaggtggaaggcattggtgtccaacggtgggctgtgctgaggtccagacatcagcctggtcaatgcattggccaccgggaggccagcgcccatgcttcctgctgccatgccccaggcctggattgcaaaatcaaggagcatgggatctcaggtcctgcagagcaggtcaccgtggcctgtgaggcaggctggaccctgactggatgcaacgtcctccctggggcattcatgactctgggggcctacgccgtggacaacatgtgtgtggcaagaagctgtgccactgacacagcaggcaggaccagtgaggaagccatagtagctgctgccatctgctgccggagccggccttcagcaaaggcctcctag

SEQ ID NO: 12 - protein, 8 aa, Artificial sequences
  Antibody sequence
GFTFSSHW

SEQ ID NO: 13 - protein, 8 aa, Artificial sequences
  Antibody sequence
INQDGSEK

SEQ ID NO: 14 - protein, 20 aa, Artificial sequences
  Antibody sequence
ARDIVLMVYDMDYYYYGMDV

SEQ ID NO: 15 - protein, 11 aa, Artificial sequences
  Antibody sequence
QSLLHSNGNNY

SEQ ID NO: 16 - protein, 3 aa, Artificial sequences
  Antibody sequence
LGS

SEQ ID NO: 17 - protein, 9 aa, Artificial sequences
  Antibody sequence
MQTLQTPLT

SEQ ID NO: 18 - protein, 127 aa, Artificial sequences
  Antibody sequence
EMQLVESGGGLVQPGGSLRLSCAASGFTFSSHWMKWVRQAPGKGLEWVANINQDGSEKYYVDSVKGRFTISRDNAKNSLFLQMNSLRAEDTAVYYCARDIVLMVYDMDYYYYGMDVWGQGTTVTVSS

SEQ ID NO: 19 - protein, 112 aa, Artificial sequences
  Antibody sequence
DIVMTQSPLSLPVTPGEPASISCRSSQSLLHSNGNNYLDWYLQKPGQSPQLLIYLGSNRASGVPDRFSGSGSGTDFTLKISRVEAEDVGVYYCMQTLQTPLTFGGGTKVEIK